(19) United States
(12) Patent Application Publication
(10) Pub. No.: US 2026/0191683 A1
(43) Pub. Date: Jul. 9, 2026

(54) SUSTAINED RELEASE OCULAR IMPLANTS

(71) Applicant: University of Tennessee Research Foundation, Memphis, TN (US)

(72) Inventors: Monica M. Jablonski, Memphis, TN (US); Mohamed Moustafa Ibrahim Moustafa, Memphis, TN (US)

(21) Appl. No.: 19/554,401

(22) Filed: Mar. 2, 2026

Related U.S. Application Data
(63) Continuation of application No. PCT/US2024/044979, filed on Sep. 3, 2024.
(60) Provisional application No. 63/579,826, filed on Aug. 31, 2023.

(51) Int. Cl. A61F 9/00; A61K 31/196; A61K 31/197

(52) U.S. Cl. CPC A61F 9/0017; A61K 31/196; A61K 31/197

(57) ABSTRACT

The present disclosure relates to the field of ocular implants suitable for sustained release of drugs (e.g., water-soluble drugs) and use of the ocular implants for treatment of ocular conditions, including glaucoma.

### 1. BACKGROUND

Glaucoma is a group of ophthalmic diseases characterized by progressive visual field loss due to damage to the optic nerve. There are four major types of adult-onset glaucoma, all of which lead to vision loss through a final pathway of retinal ganglion cell (RGC) dysfunction and/or death.

Interestingly, each form of glaucoma can be associated with multiple and sometimes divergent risk factors, indicating multiple triggering mechanisms leading to RGC demise. For three of four adult-onset glaucoma sub-types, elevated intraocular pressure (IOP) is the most significant predictive risk factor for visual field loss subsequent to RGC demise. Primary open angle glaucoma (POAG) accounts for 90% of glaucoma cases worldwide, and it is a leading cause of irreversible blindness. The fourth sub-type, normal tension glaucoma, is not associated with high IOP and factors that trigger RGC death are largely unknown. Reducing IOP has also been demonstrated to slow vision loss in patients with normal tension glaucoma.

The balance of aqueous humor (AH) production by the ciliary body (CB) and its drainage through conventional trabecular meshwork (TM) pathway, and to a lesser degree by the nonconventional uveoscleral pathway, lead to a steady-state IOP. However, in glaucoma, a high IOP is generated by an imbalance between the AH inflow and its outflow. Fortunately, IOP can be medically controlled; thus, IOP reduction is the first-line therapeutic option in glaucoma treatment.

The current standard of care for adult-onset glaucoma includes treatment with IOP-lowering medications delivered topically as eye drops. Unfortunately, this dosage form suffers from several drawbacks (e.g., rapid drainage of drug from the eye surface either to the outside of the eye or to the systemic circulation through nasolacrimal duct, and the very short corneal contact time) that may result in poor absorption and low efficacy. Further dosing can result in exaggeration of systemic side effects, lower patient satisfaction, and poor patient compliance. Additionally, glaucoma primarily affects older populations which may have difficulty with self-administration of topical drops.

Use of current daily drop medications often results in a return of elevated IOP prior to the subsequent dose, allowing for deleterious IOP fluctuations. For example, pregabalin-containing microemulsions have been found to reduce IOP by greater than 40% but IOP levels return to baseline after about 33 hours following administration in Dutch belted rabbits (Ibrahim MM, Maria DN, Mishra SR, Guragain D, Wang X, Jablonski MM. “Once Daily Pregabalin Eye Drops for Management of Glaucoma.” ACS Nano. 2019 Dec. 24; 13 (12): 13728-13744).

There is therefore a need for easily administered long-lasting formulations for treatment of ocular conditions, including glaucoma.

### 2. SUMMARY

The present disclosure relates to ocular implants that provide sustained release of a drug (e.g., a water-soluble drug or water-insoluble drug) and their use in the treatment of ocular conditions. The present disclosure also relates to methods of preparing the ocular implants described herein, and methods of using the ocular implants described herein to treat ocular conditions.

In one aspect, an ocular implant comprising a plurality of nanofibers is provided, wherein:
the nanofibers comprise a drug (e.g., a water-soluble drug, such as pregabalin) and one or more bioresorbable polymers;
the nanofibers provide for sustained release of the drug from the implant; and
the implant is suitable for implantation into the eye of a mammal.

In another aspect, an ocular implant comprising a plurality of nanoparticles is provided, wherein:
the nanoparticles comprise a drug (e.g., a water-soluble drug, such as pregabalin) and one or more bioresorbable polymers;
the nanoparticles provide for sustained release of the drug from the implant; and
the implant is suitable for implantation into the eye of a mammal.

In one aspect, a method of making an ocular implant comprising a plurality of nanofibers is provided, the method comprising:
dissolving a drug (e.g., a water-soluble drug, such as pregabalin) and one or more bioresorbable polymers in a solvent to provide a solution;
electrospinning the solution to provide a nanofiber sheet comprising a plurality of nanofibers on a collector surface;
collecting the nanofiber sheet from the collector surface; and
cutting the nanofiber sheet into a plurality of implants, each implant comprising a plurality of nanofibers; wherein
the nanofibers provide for sustained release of the drug (e.g., water-soluble drug, such as pregabalin) from the implant; and
the implant is suitable for implantation into the eye of a mammal.

In another aspect, a method of making an ocular implant comprising a plurality of nanoparticles is provided, the method comprising:
dissolving a drug (e.g., a water-soluble drug, such as pregabalin) and one or more bioresorbable polymers in a solvent to provide a solution,
electrospraying the solution to provide a plurality of nanoparticles on a collector surface (e.g., in the form of a dry powder),
collecting the plurality of nanoparticles from the collector surface, and
compressing at least a portion of the plurality of nanoparticles to form the implant; wherein
the nanoparticles provide for sustained release of the drug from the implant; and
the implant is suitable for implantation into the eye of a mammal.

In another aspect, a method of treating ocular conditions in a subject in need thereof comprises placing an implant described herein in the eye of the subject.

### 3. BRIEF DESCRIPTION OF THE DRAWINGS

These and other features, aspects, and advantages of the present disclosure will become better understood with regard to the following description, and accompanying drawings, where:

FIG. 1, panels A and B, show scanning electron microscopy (SEM) images of pregabalin nanoparticles (panel A) and pregabalin nanofibers (panel B).

FIG. 2 shows the percent of cumulative pregabalin (PRG) released from the PRG-PLA-PCL (pregabalin: DL-Lactide-co-caprolactone) implant (1 mg drug loaded) over 14 months prepared according to Example 3.

FIG. 3 shows the percent of pregabalin permeation of PRG-PLA-PCL nanofiber implants (PRG NFs, circles) and PRG-Eudragit nanoparticle implants (PRG NPs, squares) over 5 h in an ex vivo trans-scleral permeability study.

FIG. 4, panels A and B, show the swelling tests of PRG NF implants (panel A) and PRG NP implants (panel B) over 72 h.

FIG. 5 shows moisture uptake of PRG NF implants and PRG NP implants.

### 4. DETAILED DESCRIPTION

### 1. Definitions

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

As used herein, “bioresorbable polymers” refers to polymers that are naturally biodegradable when in the eye and conditions typical of the eye. In other words, bioresorbable polymers will eventually be broken down and/or absorbed by the eye. Bioresorbable polymers and their degradation products are non-toxic and possess no significant deleterious or untoward effects on the recipient's eye.

As used herein, “eye” refers to all anatomical tissues and structures comprising the eye and all anatomical tissues and structures immediately surrounding the eye. The eye is a spherical structure with a wall having three layers: the outer sclera, the middle choroid layer, and the inner retina. The sclera includes a tough fibrous coating that protects the inner layers. It is mostly white except for the transparent area at the front. The sclera and the inner surface of the eyelids are covered with a clear membrane called the conjunctiva. The space between the conjunctiva and the sclera is referred to as the subconjunctival space. The cornea is the transparent layer forming the front of the eye which allows light to enter the eye. The choroid layer, situated inside the sclera, contains many blood vessels and is modified at the front of the eye as the pigmented iris.

The biconvex lens is situated just behind the pupil. The chamber behind the lens is filled with vitreous humor, a gelatinous substance. The anterior and posterior chambers are situated between the cornea and iris, respectively, and filled with aqueous humor.

At the back of the eye is the light-detecting retina. The cornea is an optically transparent tissue that conveys images to the back of the eye. It includes avascular tissue to which nutrients and oxygen are supplied via bathing with lacrimal fluid and aqueous humor as well as from blood vessels that line the junction between the cornea and sclera. The cornea includes one pathway from the permeation of drugs into the eye.

Other anatomical tissue structures associated with the eye include the lacrimal drainage system, which includes a secretory system, a distributive system, and an excretory system. The secretory system comprises secretors that are stimulated by blinking and temperature change due to tear evaporation and reflex secretors that have an efferent parasympathetic nerve supply and secrete tears in response to physical or emotional stimulation. The distributive system includes the eyelids and the tear meniscus around the lid edges of an open eye, which spread tears over the ocular surface by blinking, thus reducing dry areas from developing.

As used herein, an “ocular implant” refers to a device that is structured, sized, or otherwise configured to be placed in an eye of a mammal. Ocular implants are generally biocompatible with physiological conditions of an eye and do not cause adverse side effects. Ocular implants may be placed in an eye without disrupting vision of the eye.

As used herein, “subject” refers to refer to animals such as mammals, including, but not limited to, primates (e.g., humans), cows, sheep, goats, horses, dogs, cats, rabbits, rats, mice, and the like. In certain embodiments, the subject is a human.

As used herein, “suitable for implantation into the eye” refers to implants that are sized, shaped, and suitably biocompatible for insertion into the tissues and structures comprising and immediately surrounding the eye as defined herein.

As used herein, “sustained release” refers to release of a drug from the ocular implant which occurs over a period which is longer than the period during which a biologically significant amount of drug would be available following direct administration of a solution of drug. Sustained release of drug can be a continuous or a discontinuous release, with relatively constant or varying rates of release. The continuity of release and level of release can be affected by the type of polymer composition used (e.g., monomer ratios, molecular weight, and varying combinations of polymers), selection of excipients, and identity and amount of drug.

“Sustained release” is also referred to in the art as “modified release,” “prolonged release,” “long-acting release (‘LAR’),” “controlled release”, or “extended release.” “Sustained release,” as used herein, also encompasses “sustained action” or “sustained effect.” “Sustained action” and “sustained effect,” as those terms are used herein, refer to an increase in the time period over which a drug performs its therapeutic or prophylactic activity as compared to an appropriate control. “Sustained action” is also known to those experienced in the art as “prolonged action” or “extended action.”

In certain embodiments, the sustained release profile of the ocular implants described herein exhibit an initial burst release of drug followed by a slower sustained release of the drug following administration.

Without being bound by a particular theory, it is believed that the release of the drug can occur by two different mechanisms. First, the drug can be released by diffusion through the polymer matrix of the implant. A second mechanism is the release of the drug due to degradation of the bioresorbable polymer.

As used herein, “treat”, “treating”, or “treatment” refers to reduction, resolution, or prevention of an ocular condition, ocular injury, or ocular damage or to promotion of healing of injured or damaged ocular tissue. A treatment is usually effective to reduce at least one symptom of an ocular condition, ocular injury, or ocular damage.

As used herein, “swelling” refers to the process of absorbing water to increase the volume of a polymer network after insertion at the target insertion site that helps for drug release from areas of the implant, e.g., deeper areas of the implant.

As used herein, “swelling index” refers to the swelling percentage calculated according to the following equation:

$$swelling percentage = [ ( weight of swollen implant - weight of initial implant ) / weight of initial implant ] \times 100.$$

As used herein, “moisture uptake” refers to the ability of an implant to absorb moisture from the atmosphere during their storage at shelf life, resulting in the diffusion and sorption by the polymer matrix, and the progressive passage and filling of the cavities by water. As used in the present disclosure, moisture uptake can be determined using the following equation:

$$moisture uptake ( \% ) = [ ( final weight of implant - initial weight of implant ) / initial weight of implant ] \times 100.$$

As used herein, “shelf stability” or “shelf-stable” refers to the ability of an implant to demonstrate less than 10% (or, if explicitly stated, less than 9%, less than 8%, less than 7%, less than 6%, less than 5%, less than 4%, less than 3%, less than 2%, or less than 1%) degradation of the drug after storage for 4 weeks (or, if explicitly stated, for 3, 6, or 12 months) upon storage at ambient conditions.

As used herein, “relative humidity” (RH) refers to the ratio of the partial pressure of water vapor in air to the saturation vapor pressure of water at the same temperature, expressed as a percentage.

As used herein, “protein fouling” refers to the formation of protein aggregates adhering to the surface.

As used herein, “permeability” refers to the velocity at which a molecule crosses a membrane and indicates how easy it is for the molecule to move across the membrane in a direction normal to the membrane surface.

As used herein, “rate of permeation” refers to rate of drug transfer across a membrane barrier (amount of drug per time unit, dM/dt).

As used herein, “flux” of an active agent is the rate of penetration of the active agent per unit surface area of a membrane. In some instances, the flux can be determined by the equation:

$$Flux ( J ) = ( dM / dt ) / A$$

where J is the flux, dM/dt is the rate of permeation across a membrane, A is the membrane surface area.

As used herein, “permeability coefficient” refers to the measure of permeability as defined in the present disclosure. In some instances, the permeability coefficient can be determined by the equation:

$$P = J / {C}_{0}$$

where P is the permeability coefficient, J is the flux, and Co is the initial drug concentration of the implant.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The present disclosure additionally encompasses compounds described herein as individual isomers substantially free of other isomers, and alternatively, as mixtures of various isomers.

### 2. OCULAR IMPLANTS

In one aspect, the present disclosure provides ocular implants that provide sustained release of a drug (e.g., a water-soluble drug, such as pregabalin) formulated within the implant. The implants described herein provide sustained drug release over several months and are composed of one or more bioresorbable polymers. As such, spent implants do not require removal, surgical or otherwise.

### 2.1 Nanofiber Implants

In certain embodiments, an ocular implant is provided, the ocular implant comprising a plurality of nanofibers, wherein:
the nanofibers comprise a drug (e.g., a water-soluble drug, such as pregabalin) and one or more bioresorbable polymers;
the nanofibers provide for sustained release of the drug from the implant; and
the implant is suitable for implantation into the eye of a mammal.

In certain embodiments, the drug is selected from an antibacterial, an antiviral, antifungal, a steroid, a sterol, an anti-inflammatory, an angiogenesis inhibitor, a growth factor, an anti-coagulant, an antioxidant, a pyridine, an antihypertensive, an antidiabetic, an insulin, a progenitor/stem cell, an intraocular pressure (IOP) lowering drug, and a combination thereof.

In certain embodiments, the drug is an antibacterial. Exemplary antibacterial and/or antibiotic drugs include, but are not limited to, amikacin, amoxicillin, clavulanic acid, ampicillin, benzathine benzylpenicillin, benzylpenicillin, cefalexin, cefazolin, chloramphenicol, clindamycin, cloxacillin, doxycycline, gentamicin, metronidazole, nitrofurantoin, phenoxymethylpenicillin, procaine benzylpenicillin, spectinomycin, sulfamethoxazole, trimethoprim, azithromycin, cefixime, cefotaxime, ceftriaxone, cefuroxime, ciprofloxacin, delafloxacin, gemifloxacin, levofloxacin, moxifloxacin, norfloxacin, ofloxacin, clarithromycin, piperacillin, tazobactam, vancomycin, cefiderocol, ceftazidime, avibactam, ceftolozane, colistin, fosfomycin, linezolid, meropenem, vaborbactam, plazomicin, polymyxin B, pharmaceutically acceptable salts of the foregoing, and combinations thereof.

In certain embodiments, the drug is an antiviral. Exemplary antivirals include, but are not limited to, acyclovir, valaciclovir, abacavir, lamivudine, tenofovir, tenofovir disoproxil fumarate, zidovudine, efavirenz, atazanavir, ritonavir, darunavir, lopinavir, dolutegravir, raltegravir, emtricitabine, isoniazid, pyridoxine, sulfamethoxazole, trimethoprim, ribavirin, ganciclovir, valganciclovir, oseltamivir, entecavir, daclatasvir, sofosbuvir, glecaprevir, pibrentasvir, ravidasvir, velpatasvir, ledipasvir, pharmaceutically acceptable salts of the foregoing, and combinations thereof.

In certain embodiments, the drug is an antifungal. Exemplary antifungal drugs include, but are not limited to, clotrimazole, econazole. miconazole. terbinafine, fluconazole, ketoconazole, nystatin, amphotericin, and combinations thereof.

In certain embodiments, the drug is a steroid, e.g., corticosteroid. Exemplary corticosteroid drugs include, but are not limited to, betamethasone, prednisone, prednisolone, methylprednisolone, dexamethasone, hydrocordison, cortisone, ethamethasoneb, triamcinolone, fludrocortisone, pharmaceutically acceptable salts of the foregoing, and combinations thereof.

In certain embodiments, the drug is a sterol. Exemplary sterol drugs include, but are not limited to, cholesterol, mycosterol, such as ergosterol, phytosterol, such as campesterol, sitosterol, stigmasterol, pharmaceutically acceptable salts of the foregoing, and combinations thereof.

In certain embodiments, the drug is an anti-inflammatory. Exemplary anti-inflammatory drugs include, but are not limited to, aspirin, ibuprofen, naproxen sodium, celecoxib, diclofenac, fenoprofen, indomethacin, ketorolac, pharmaceutically acceptable salts of the foregoing, and combinations thereof.

In certain embodiments, the drug is an angiogenesis inhibitor, e.g., anti-VEGF. Exemplary angiogenesis inhibitors include, but are not limited to, pegaptanib, bevacizumab, ranibizumab, sorafenib, dasatinib, sunitinib, nilotinib, pazopanib, aflibercept, pharmaceutically acceptable salts of the foregoing, and combinations thereof.

In certain embodiments, the drug is a growth factor. Exemplary growth factors include, but are not limited to, Erthropoiesis stimulating agents (e.g., ancestim, epoetin alfa, darbepoetin alfa, methoxy polyethylene glycol-epoetin beta, and luspatercept), granulocyte colony stimulating factor (e.g., filgrastim, pegfilgrastim, eflapegrastim, and oprelvekin), granulocyte-macrophage colony stimulating factor (e.g., sargramostim), thrombopoiesis-stimulating agents (e.g., such as romiplostim and eltrombopag), pharmaceutically acceptable salts of the foregoing, and combinations thereof.

In certain embodiments, the drug is an anti-coagulant. Exemplary anti-coagulants include, but are not limited to, heparin, rivaroxaban, warfarin, apixaban, argatroban, bivalrudin, pharmaceutically acceptable salts of the foregoing, and combinations thereof.

In certain embodiments, the drug is an antioxidant. Exemplary antioxidants include, but are not limited to, tocopherols, ascorbic acid, rosemary extract, butylated hydroxyanisole, butylated hydroxytoluene, propyl gallate, vitamin C, beta-carotene, selenium, alpha lipoid acid, lycopene, resveratrol, pharmaceutically acceptable salts of the foregoing, and combinations thereof.

In certain embodiments, the drug is a pyridine. Exemplary pyridine drugs include, but are not limited to, antimicrobial agents (e.g., isoniazid and ethionamide), antiviral drugs (e.g., nevirapine, tipranavir, and doravirine), anticancer drugs (e.g., acalabrutinib, neratinib and abemaciclib), cardiovascular drugs (e.g., nimodipine and nifedipine), proton pump inhibitors (e.g., esomeprazole and lansoprazole), antiasthma drugs (e.g., montelukast), antidiabetic drugs (e.g., pioglitazone), pharmaceutically acceptable salts of the foregoing, and combinations thereof.

In certain embodiments, the drug is a antihypertensive. Exemplary antihypertensive drugs include, but are not limited to, diuretics (e.g., furosemide, bumetanide, torsemide, chlorothiazide, amiloride, hydrochlorothiazide, indapamide, metolazone, and triamterene), beta-blockers (e.g., acebutolol, atenolol, betaxolol, carvedilol, carvedilol phosphate, labetalol, metoprolol succinate, metoprolol tartrate, nadolol, nebivolol, pindolol, and propranolol), angiotensin-converting enzyme inhibitors (e.g., captopril, enalapril, fosinopril, lisinopril, perindopril, quinapril, ramipril, trandolapril, benazepril, and moexipril), angiotensin II receptor blockers (e.g., candesartan, losartan, and valsartan), calcium channel blockers (e.g., amlodipine, diltiazem, felodipine, isradipine, nicardipine, nifedipine, nisoldipine, and verapamil), alpha blockers (e.g., doxazosin, prazosin, and terazosin hydrochloride), alpha-2 receptor agonists (e.g., methyldopa, clonidine, and guanfacine), combined alpha and beta blockers (e.g., carvedilol and labetalol hydrochloride), vasodilators (e.g., hydralazine and minoxidil; pharmaceutically acceptable salts of any of the foregoing, and combinations thereof.

In certain embodiments, the drug is an antidiabetic. Exemplary antidiabetic drugs include, but are not limited to, sulfonylureas (e.g., glipizide, glyburide, gliclazide, and glimepiride), meglitinides (e.g., repaglinide and nateglinide), biguanides (e.g., metformin), thiazolidinediones (e.g., rosiglitazone and pioglitazone), α-Glucosidase inhibitors (e.g., acarbose, miglitol, and voglibose), DPP-4 inhibitors (e.g., sitagliptin, saxagliptin, vildagliptin, linagliptin, and alogliptin), SGLT2 inhibitors (e.g., dapagliflozin and canagliflozin), Cycloset (e.g., bromocriptine), GLP-1 receptor agonists (e.g., semaglutide, Exenatide, Liraglutide, and Lixisenatide), curcumin, pharmaceutically acceptable salts of the foregoing, and combinations thereof.

In certain embodiments, the drug is an insulin. Exemplary insulins include, but are not limited to, rapid-acting insulin (e.g., insulin lispro), short-acting insulin (e.g., regular human insulin), intermediate-acting insulin (e.g., NPH insulin), long-acting insulin (e.g., insulin detemir, insulin glargine, insulin glargine-yfgn, and insulin degludec), and insulin mixtures (e.g., NPH/regular, protamine/lispro, or protamine/aspart).

In certain embodiments, the drug is stem cells. Exemplary stem cells include, but are not limited to, embryonic stem cells (pluripotent stem cells), adult stem cells (e.g., hematopoietic stem cells), mesenchymal stem cells (e.g., umbilical cord tissue-derived mesenchymal stem cells), and induced pluripotent stem cells (iPSCs).

In certain embodiments, the drug lowers intraocular pressure. Exemplary drugs that lower intraocular pressure include, but are not limited to, CACNA2D1 inhibitors, CACNA2D2 inhibitors, gabapentenoids (e.g., pregabalin and gabapentin), calcium channel blockers (e.g., nimodipine, amlodipine, diltiazem, felodipine, isradipine, nicardipine, nifedipine, nisoldipine, and verapamil), cholinergics and adrenergics (e.g., pilocarpine, carbachol, physostigmine, neostigmine and ecothiophate), adrenergic agonists (e.g., epinephrine and dipivefrin), beta (β)-blockers (e.g., timolol, levobunolol, metipranolol, carteolol, and betaxolol), carbonic anhydrase inhibitors (e.g., brinzolamide, dorzolamide, methazolamide, and acetazolamide), alpha agonist clonidine derivatives (e.g., brimonidine tartrate and apraclonidine), prostaglandin analogs (e.g., bimatoprost, travoprost, latanoprost and tafluprost), rho-kinase inhibitors (e.g., netarsudil), pharmaceutically acceptable salts of the foregoing, and combinations thereof.

In certain embodiments, the drug is a water-soluble drug. In certain embodiments, the water-soluble drug is selected from beta-blockers (e.g., betaxolol and timolol), prostaglandin analogs (e.g., bimatoprost, latanoprost, and travoprost), alpha-adrenergic agents (e.g., brimonidine tartrate), carbonic anhydrase inhibitors (e.g., brinzolamide, dorzolamide, and acetazolamide), calcium channel blockers (e.g., nimodipine and pregabalin), asialo, galactosylated, triantennary (NA3) (asialo-, tri-antennary complex-type N-glycan), OT-551 (hydrochloride (1-hydroxy-2,2,6,6-tetramethyl-4-piperidinyl cyclopropane carboxylic acid ester hydrochloride), brimonidine tartrate, clindamycin, ciprofloxacin, levofloxacin, gatifloxacin, gemifloxacin, ofloxacin, triamcinolone, valacyclovir, pyrimethamine, valganciclovir, ganciclovir, acyclovir, foscarnet, prednisolone acetate, diflupednate, triamcinolone, dexamethasone, methotrexate, azathioprine, mycophenolate mofetil, cyclosporine, tacrolimus, cyclophosphamide, ribavirin, bromfenac, ketorolac, nepafenac, lifitegrast, flubiprofen, diclonfenac, ketotifen, nedocromil, phenylephrine, azelastine, epinastine, naphazoline/pheniramine, oloptadine, bepotastine, alacaftadine, pemirolast, tetrahydrozoline with or without zinc sulfate, iodoxamide, naphazoline, phenylephrine, cromolyn, emedastine, oxymetazoline, xylometazoline, loratidine, desloratidine, phenylglycine, gabapentin, a pharmaceutically acceptable salt thereof, and a combination thereof.

In certain embodiments, the drug is a water-insoluble drug.

In certain embodiments, the implant comprises from 0.1 wt to 50 wt % of the drug (e.g., water-soluble drug or water-insoluble drug), such as, for example, from 0.1 wt % to 40 wt %, 0.1 wt % to 30 wt %, from 0.1 wt % to 20 wt %, from 0.1 wt % to 10 wt %, from 0.1 wt % to 5 wt %, from 1 wt % to 50 wt %, from 1 wt % to 40 wt %, from 1 wt % to 30 wt %, from 1 wt % to 20 wt %, from 1 wt % to 10 wt %, from 1 wt % to 5 wt %, from 5 wt % to 50 wt %, from 5 wt % to 40 wt %, from 5 wt % to 30 wt %, from 5 wt % to 20 wt %, from 5 wt % to 10 wt %, from 10 wt % to 50 wt %, from 10 wt % to 40 wt %, from 10 wt % to 30 wt %, from 10 wt % to 20 wt %, from 20 wt % to 50 wt %, from 20 wt % to 40 wt %, from 20 wt % to 30 wt %, from 30 wt % to 50 wt %, from 30 wt % to 40 wt %, or from 40 wt % to 50 wt %. In certain embodiments, the implant comprises about 20 wt % of the drug (e.g., water-soluble drug).

In certain embodiments, the implant comprises from 0.1 mg to 20 mg of the drug (e.g., water-soluble drug or water-insoluble drug), such as, for example, from 0.1 mg to 15 mg, from 0.1 mg to 10 mg, from 0.1 mg to 5 mg, from 0.1 mg to 2.5 mg, from 1 mg to 20 mg, from 1 mg to 15 mg, from 1 mg to 10 mg, from 1 mg to 5 mg, from 1 mg to 2.5 mg, from 2.5 mg to 20 mg, from 2.5 mg to 15 mg, from 2.5 mg to 10 mg, from 2.5 mg to 5 mg, from 5 mg to 20 mg, from 5 mg to 15 mg, from 5 mg to 10 mg, from 10 mg to 20 mg, from 10 mg to 15 mg, or from 15 mg to 20 mg. In certain embodiments, the implant comprises from 0.1 mg to 5 mg of the drug, such as, for example, from 0.1 mg to 4 mg, from 0.1 mg to 3 mg, from 0.1 mg to 2 mg from 0.1 mg to 1 mg, from 0.5 mg to 5 mg, from 0.5 mg to 4 mg, from 0.5 mg to 3 mg, from 0.5 mg to 2 mg, from 0.5 mg to 1 mg, from 0.5 mg to 1 mg, from 1 mg to 5 mg, from 1 mg to 4 mg, from 1 mg to 3 mg, from 1 mg to 2 mg, from 2 mg to 5 mg, from 2 mg to 4 mg, from 2 mg to 3 mg, from 3 mg to 5 mg, from 3 mg to 4 mg, and from 4 mg to 5 mg. In certain embodiments, the implant comprises from 0.5 mg to 2 mg of the drug (e.g., water-soluble drug).

In certain embodiments, the drug is a calcium channel blocker. In certain embodiments, the drug is pregabalin. In certain embodiments, the implant comprises from 0.1 mg to 5 mg of pregabalin, such as, for example, from 0.1 mg to 4 mg, from 0.1 mg to 3 mg, from 0.1 mg to 2 mg from 0.1 mg to 1 mg, from 0.5 mg to 5 mg, from 0.5 mg to 4 mg, from 0.5 mg to 3 mg, from 0.5 mg to 2 mg, from 0.5 mg to 1 mg, from 0.5 mg to 1 mg, from 1 mg to 5 mg, from 1 mg to 4 mg, from 1 mg to 3 mg, from 1 mg to 2 mg, from 2 mg to 5 mg, from 2 mg to 4 mg, from 2 mg to 3 mg, from 3 mg to 5 mg, from 3 mg to 4 mg, and from 4 mg to 5 mg. In certain embodiments, the implant comprises from 0.5 mg to 2 mg of pregabalin.

In certain embodiments, the one or more bioresorbable polymers comprise poly(alpha-hydroxy acids), poly(lactide-co-glycolide) (PLG), poly(DL-lactide-co-caprolactone) (DL-PLCL), polycaprolactone (PCL), poly(L-lactic acid) (PLA), poly(trimethylene carbonate) (PTMC), polydioxanone (PDO), poly(4-hydroxy butyrate) (PHB), polyhydroxyalkanoates (PHA), poly(phosphazene), polyphosphate ester, poly(amino acid), polydepsipeptides, poly(butylene succinate) (PBS), polyethylene oxide, polypropylene fumarate, polyiminocarbonates, poly(lactide-co-caprolactone) (PLCL), poly(glycolide-co-caprolactone) (PGCL) copolymer, poly(D,L-lactic acid), polyglycolic acid, poly(L-lactide-co-D,L-lactide), poly(L-lactide-co-glycolide), poly(D,L-lactide-co-glycolide), poly(gycolide-trimethylene carbonate), poly(glycolide-co-caprolactone) (PGCL), poly(ethyl glutamate-co-glutamic acid), poly(tert-butyloxy-carbonylmethyl glutamate), poly(glycerol sebacate), tyrosine-derived polycarbonate, poly 1,3-bis-(p-carboxyphenoxy) hexane-co-sebacic acid, polyphosphazene, ethyl glycinate polyphosphazene, polycaprolactone co-butylacrylate, a copolymer of polyhydroxybutyrate, a copolymer of maleic anhydride, a copolymer of poly(trimethylene carbonate), polyethylene glycol (PEG), hydroxypropylmethylcellulose and cellulose derivatives, polysaccharides (e.g., hyaluronic acid, chitosan and starch), proteins (e.g., gelatin and collagen), polyaspirins, polyphosphagenes, collagen, alginates, albumin, fibrin, vitamin E analogs, D-lactide, D,L-lactide, L-lactide, D,L-lactide-caprolactone (DL-CL), D,L-lactide-glycolide-caprolactone (DL-G-CL), dextrans, vinylpyrrolidone, polyvinyl alcohol (PVA), PVA-g-PLGA, PEGT-PBT copolymer (polyactive), methacrylates, poly(N-isopropylacrylamide), PEO—PPO-PEO (pluronics), PEO—PPO-PAA copolymers, PLGA-PEO-PLGA, PEG-PLG, PLA-PLGA, poloxamer 407, PEG-PLGA-PEG triblock copolymers, SAIB (sucrose acetate isobutyrate) hydroxypropyl cellulose, hydroxypropyl methylcellulose, hydroxyethyl methylcellulose, salts thereof, Carbopol®, carboxymethylcellulose or poly(methoxyethylmethacrylate), poly(hydroxyethylmethacrylate), poly(methoxyethoxy-ethylmethacrylate), polymethylmethacrylate (PMMA), methylmethacrylate (MMA), gelatin, polyvinyl alcohols, propylene glycol, or combinations thereof.

In certain embodiments, the one or more bioresorbable polymers comprise poly(L-lactic acid) (PLA), polycaprolactone (PCL), polyglycolic acid, or a copolymer thereof. In certain embodiments, the one or more bioresorbable polymers comprise poly(L-lactic acid) (PLA). In certain embodiments, the one or more bioresorbable polymers comprise polycaprolactone (PCL). In certain embodiments, the one or more bioresorbable polymers comprise poly(L-lactic acid) (PLA). In certain embodiments, the one or more bioresorbable polymers comprise a copolymer of poly(L-lactic acid) (PLA), polycaprolactone (PCL), and polyglycolic acid.

In certain embodiments, the implant comprises from 50 wt % to 99.9 wt % of the one or more bioresorbable polymers, such as, for example, from 60 wt % to 99.9 wt %, from 70 wt % to 99.9 wt %, from 80 wt % to 99.9 wt %, from 80 wt % to 99.9 wt %, or from 90 wt % to 99.9 wt %. In certain embodiments, the implant comprises from 75 wt % to 99.9 wt % of the one or more bioresorbable polymers, such as, for example, from 75 wt % to 95 wt %, from 80 wt % to 95 wt %, from 85 wt % to 95 wt %, or from 90 wt % to 95 wt %. In certain embodiments, the implant comprises 80 wt % of the one or more bioresorbable polymers.

In certain embodiments, the weight ratio of drug (e.g., water-soluble drug) to one or more bioresorbable polymer is from 1:1 to 1:20, such as, for example, from 1:1 to 1:15, from 1:1 to 1:10, from 1:1 to 1:5, from 1:2 to 1:6, from 1:2 to 1:5, from 1:2 to 1:4, from 1:2 to 1:3, from 1:3 to 1:6, from 1:3 to 1:5, from 1:3 to 1:4, from 1:4 to 1:6, or from 1:4 to 1:5. In certain embodiments, the weight ratio of drug (e.g., water-soluble drug) to one or more bioresorbable polymer is 1:4.

In certain embodiments, the nanofibers further comprise a permeation enhancer. In certain embodiments, the permeation enhancer is selected from D-α-Tocopherol polyethylene glycol succinate (TPGS), dioctyl sodium sulfosuccinate, sodium caprate, sodium N-[8 (−2-hydroxybenzoyl)amino]caprylate (SNAC), sodium lauryl sulfate, sodium salicylate, oleic acid, lecithin, dehydrated alcohol, polysorbates or polyoxyethylene sorbitan fatty acids esters (Tweens), sorbitan fatty acids esters (Spans), polyoxyethylene alkyl ether (Brijs), polyoxyl 40 stearate, polyoxy ethylene 50 stearate, polyethylene glycol, polyvinyl alcohol, polyvinylpyrrolidone (e.g., polyvinylpyrrolidone K29-32), hydroxy propyl methyl cellulose, polyvinylpyrrolidone/vinyl acetate (VP/VA) copolymer, poly(lactic-co-glycolic acid), edetate disodium, propylene glycol, glycerol monooleate, bile salts, octoxynol, non-ionic surfactants, anionic surfactants, cationic surfactants, and a combination thereof.

In certain embodiments, the implant has one of the following shapes: spherical, oblate, ellipsoidal, rod, disc, tube, hemispherical, oval, cylindrical, or irregular. In certain embodiments, the implant is rod-shaped.

In certain embodiments, the implant has the following dimensions: from 0.5 mm to 6 mm in length, from 0.5 mm to 6 mm in width, and from 0.2 mm to 6 mm in height. With respect to length, the implant can be from 0.5 mm to 5 mm, from 0.5 mm to 4 mm, from 0.5 mm to 3 mm, 0.5 mm to 2 mm, 0.5 mm to 1 mm, 1 mm to 6 mm, 1 mm to 5 mm, 1 mm to 4 mm, 1 mm to 3 mm, 1 mm to 2 mm, 2 mm to 6 mm, 2 mm to 5 mm, 2 mm to 4 mm, 2 mm to 3 mm, 3 mm to 6 mm, 3 mm to 5 mm, 3 mm to 4 mm, 4 mm to 6 mm, 4 mm to 5 mm, or 5 mm to 6 mm. With respect to width, the implant can be from 0.5 mm to 5 mm, from 0.5 mm to 4 mm, from 0.5 mm to 3 mm, 0.5 mm to 2 mm, 0.5 mm to 1 mm, 1 mm to 6 mm, 1 mm to 5 mm, 1 mm to 4 mm, 1 mm to 3 mm, 1 mm to 2 mm, 2 mm to 6 mm, 2 mm to 5 mm, 2 mm to 4 mm, 2 mm to 3 mm, 3 mm to 6 mm, 3 mm to 5 mm, 3 mm to 4 mm, 4 mm to 6 mm, 4 mm to 5 mm, or 5 mm to 6 mm. With respect to height, the implant can be from 0.2 mm to 6 mm, 0.2 mm to 5 mm, 0.2 mm to 4 mm, 0.2 mm to 3 mm, 0.2 mm to 2 mm, 0.2 mm to 1 mm, 0.5 mm to 6 mm, 0.5 mm to 5 mm, from 0.5 mm to 4 mm, from 0.5 mm to 3 mm, 0.5 mm to 2 mm, 0.5 mm to 1 mm, 1 mm to 6 mm, 1 mm to 5 mm, 1 mm to 4 mm, 1 mm to 3 mm, 1 mm to 2 mm, 2 mm to 6 mm, 2 mm to 5 mm, 2 mm to 4 mm, 2 mm to 3 mm, 3 mm to 6 mm, 3 mm to 5 mm, 3 mm to 4 mm, 4 mm to 6 mm, 4 mm to 5 mm, or 5 mm to 6 mm. In certain embodiments, the implant is 2 to 6 mm in length, 1 to 3 mm in width, and 0.2 mm to 1 mm in height.

In certain embodiments, the nanofibers of the implant have a diameter from 100 nm to 1,000 nm, such as, for example, from 100 nm to 750 nm, from 100 nm to 500 nm, from 100 nm to 250 nm, from 250 nm to 1,000 nm, from 250 nm to 750 nm, from 250 nm to 500 nm, from 500 nm to 1,000 nm, from 500 nm to 750 nm, or from 750 nm to 1,000 nm. In certain embodiments, the nanofibers of the implant have a diameter from 200 nm to 500 nm, from 200 nm to 400 nm, or from 300 nm to 350 nm.

In certain embodiments, the implant weighs from 0.5 mg to 10 mg, such as, for example, from 0.5 mg to 7.5 mg from 0.5 mg to 5 mg, from 0.5 mg to 2.5 mg, from 2.5 mg to 10 mg, from 2.5 mg to 7.5 mg, from 2.5 mg to 5 mg, from 5 mg to 10 mg, from 5 mg to 7.5 mg, or from 7.5 mg to 10 mg. In certain embodiments, the implant weighs from 3 mg to 8 mg, from 3 mg to 6 mg or from 4 mg to 6 mg. In certain embodiments, the implant weighs 5 mg.

In some embodiments, when placed in room temperature water for 2 h the implant gives the water a pH from 5 to 8, such as, for example, from 6 to 8, from 6 to 7, from 7 to 8, or from 6.5 to 7.5.

In certain embodiments, an ocular implant comprises a plurality of nanofibers, wherein:
the nanofibers comprise a drug (e.g., water-soluble drug) and one or more bioresorbable polymers, wherein the one or more bioresorbable polymers comprise poly(L-lactic acid) (PLA), polycaprolactone (PCL), polyglycolic acid, or a copolymer thereof;
the nanofibers provide for sustained release of the drug from the implant; and
the implant is suitable for implantation into the eye of a mammal.

In certain embodiments, an ocular implant comprises a plurality of nanofibers, wherein:
the nanofibers comprise a drug (e.g., water-soluble drug) and one or more bioresorbable polymers, wherein the drug is pregabalin and the one or more bioresorbable polymers comprise poly(L-lactic acid) (PLA), polycaprolactone (PCL), polyglycolic acid, or a copolymer thereof;
the nanofibers provide for sustained release of the drug from the implant; and
the implant is suitable for implantation into the eye of a mammal.

In certain embodiments, an ocular implant comprises a plurality of nanofibers, wherein:
the nanofibers comprise a drug (e.g., water-soluble drug) and one or more bioresorbable polymers, wherein the one or more bioresorbable polymers comprise poly(L-lactic acid) (PLA), polycaprolactone (PCL), polyglycolic acid, or a copolymer thereof, wherein the weight ratio of drug to one or more bioresorbable polymers is from 1:1 to 1:20, preferably from 1:1 to 1:10, more preferably from 1:1 to 1:6, still more preferably 1:4;
the nanofibers provide for sustained release of the drug from the implant; and
the implant is suitable for implantation into the eye of a mammal.

In certain embodiments, an ocular implant comprises a plurality of nanofibers, wherein:
the nanofibers comprise a drug and one or more bioresorbable polymers, wherein the drug is pregabalin and the one or more bioresorbable polymers comprise poly(L-lactic acid) (PLA), polycaprolactone (PCL), polyglycolic acid, or a copolymer thereof, wherein the weight ratio of pregabalin to one or more bioresorbable polymers is from 1:1 to 1:20, preferably from 1:1 to 1:10, more preferably from 1:1 to 1:6, still more preferably 1:4;
the nanofibers provide for sustained release of the drug from the implant; and
the implant is suitable for implantation into the eye of a mammal.

In certain embodiments, an ocular implant comprises a plurality of nanofibers, wherein:
the nanofibers comprise a drug (e.g., water-soluble drug) and one or more bioresorbable polymers, wherein the one or more bioresorbable polymers comprise poly(L-lactic acid) (PLA), polycaprolactone (PCL), polyglycolic acid, or a copolymer thereof, or a copolymer thereof;
the nanofibers provide for sustained release of the drug from the implant;
the implant is suitable for implantation into the eye of a mammal; and
the implant comprises from 0.1 mg to 5 mg drug.

In certain embodiments, an ocular implant comprises a plurality of nanofibers, wherein:
the nanofibers comprise a drug and one or more bioresorbable polymers, wherein the drug is pregabalin and the one or more bioresorbable polymers comprise poly(L-lactic acid) (PLA), polycaprolactone (PCL), polyglycolic acid, or a copolymer thereof, or a copolymer thereof;
the nanofibers provide for sustained release of the drug from the implant;
the implant is suitable for implantation into the eye of a mammal; and
the implant comprises from 0.1 mg to 5 mg pregabalin.

In certain embodiments, an ocular implant comprises a plurality of nanofibers, wherein:
the nanofibers comprise from 0.1 wt % to 50 wt % of a drug (e.g., water-soluble drug) and from 50 wt % to 99.9 wt % of one or more bioresorbable polymers, wherein the one or more bioresorbable polymers comprise poly(L-lactic acid) (PLA), polycaprolactone (PCL), polyglycolic acid, or a copolymer thereof;
the nanofibers provide for sustained release of the drug from the implant; and
the implant is suitable for implantation into the eye of a mammal.

In certain embodiments, an ocular implant comprises a plurality of nanofibers, wherein:
the nanofibers comprise from 0.1 wt % to 50 wt % of a drug and from 50 wt % to 99.9 wt % of one or more bioresorbable polymers, wherein the drug is pregabalin and the one or more bioresorbable polymers comprise poly(L-lactic acid) (PLA), polycaprolactone (PCL), polyglycolic acid, or a copolymer thereof;
the nanofibers provide for sustained release of the drug from the implant; and
the implant is suitable for implantation into the eye of a mammal.

In certain embodiments, an ocular implant comprises a plurality of nanofibers, wherein:
the nanofibers comprise from 1% to 30 wt % of a drug (e.g., water-soluble drug) and from 75 wt % to 95 wt % of one or more bioresorbable polymers, wherein the one or more bioresorbable polymers comprises poly(L-lactic acid) (PLA), polycaprolactone (PCL), polyglycolic acid, or a copolymer thereof;
the nanofibers provide for sustained release of the drug from the implant; and
the implant is suitable for implantation into the eye of a mammal.

In certain embodiments, an ocular implant comprises a plurality of nanofibers, wherein:
the nanofibers comprise from 1% to 30 wt % of a drug and from 75 wt % to 95 wt % of one or more bioresorbable polymers, wherein the drug is pregabalin and the one or more bioresorbable polymers comprises poly(L-lactic acid) (PLA), polycaprolactone (PCL), polyglycolic acid, or a copolymer thereof;
the nanofibers provide for sustained release of the drug from the implant; and
the implant is suitable for implantation into the eye of a mammal.

In certain embodiments, an ocular implant comprises a plurality of nanofibers, wherein:
the nanofibers comprise 20 wt % of a drug (e.g., water-soluble drug) and from 80 wt % of one or more bioresorbable polymers, wherein the one or more bioresorbable polymers comprises poly(L-lactic acid) (PLA), polycaprolactone (PCL), polyglycolic acid, or a copolymer thereof;
the nanofibers provide for sustained release of the drug from the implant; and
the implant is suitable for implantation into the eye of a mammal.

In certain embodiments, an ocular implant comprises a plurality of nanofibers, wherein:
the nanofibers comprise 20 wt % of a drug and from 80 wt % of one or more bioresorbable polymers, wherein the drug is pregabalin and the one or more bioresorbable polymers comprises poly(L-lactic acid) (PLA), polycaprolactone (PCL), polyglycolic acid, or a copolymer thereof;
the nanofibers provide for sustained release of the drug from the implant; and
the implant is suitable for implantation into the eye of a mammal.

### 2.2 Nanoparticle Implants

In certain embodiments, an ocular implant is provided, the ocular implant comprising a plurality of nanoparticles, wherein:
the nanoparticles comprise a drug (e.g., water-soluble drug) and one or more bioresorbable polymers;
the nanoparticles provide for sustained release of the drug from the implant; and
the implant is suitable for implantation into the eye of a mammal.

In certain embodiments, the drug is as described herein above, i.e., selected from an antibacterial, an antiviral, antifungal, a steroid, a sterol, an anti-inflammatory, an angiogenesis inhibitor, a growth factor, an anti-coagulant, an antioxidant, a pyridine, an antihypertensive, an antidiabetic, an insulin, a progenitor/stem cell, an intraocular pressure (IOP) lowering drug, and a combination thereof.

In certain embodiments, an ocular implant is provided, the ocular implant comprising a plurality of nanoparticles, wherein:
the nanoparticles comprise pregabalin and one or more bioresorbable polymers;
the nanoparticles provide for sustained release of the pregabalin from the implant; and
the implant is suitable for implantation into the eye of a mammal.

In certain embodiments, the implant comprises from 0.1 wt to 50 wt % of the drug (e.g., water-soluble drug, such as pregabalin), such as, for example, from 0.1 wt % to 40 wt %, 0.1 wt % to 30 wt %, from 0.1 wt % to 20 wt %, from 0.1 wt % to 10 wt %, from 0.1 wt % to 5 wt %, from 1 wt % to 50 wt %, from 1 wt % to 40 wt %, from 1 wt % to 30 wt %, from 1 wt % to 20 wt %, from 1 wt % to 10 wt %, from 1 wt % to 5 wt %, from 5 wt % to 50 wt %, from 5 wt % to 40 wt %, from 5 wt % to 30 wt %, from 5 wt % to 20 wt %, from 5 wt % to 10 wt %, from 10 wt % to 50 wt %, from 10 wt % to 40 wt %, from 10 wt % to 30 wt %, from 10 wt % to 20 wt %, from 20 wt % to 50 wt %, from 20 wt % to 40 wt %, from 20 wt % to 30 wt %, from 30 wt % to 50 wt %, from 30 wt % to 40 wt %, or from 40 wt % to 50 wt %. In certain embodiments, the implant comprises about 20 wt % of the drug (e.g., water-soluble drug, such as pregabalin).

In certain embodiments, the implant comprises from 0.1 mg to 20 mg of the drug (e.g., water-soluble drug, such as pregabalin), such as, for example, from 0.1 mg to 15 mg, from 0.1 mg to 10 mg, from 0.1 mg to 5 mg, from 0.1 mg to 2.5 mg, from 1 mg to 20 mg, from 1 mg to 15 mg, from 1 mg to 10 mg, from 1 mg to 5 mg, from 1 mg to 2.5 mg, from 2.5 mg to 20 mg, from 2.5 mg to 15 mg, from 2.5 mg to 10 mg, from 2.5 mg to 5 mg, from 5 mg to 20 mg, from 5 mg to 15 mg, from 5 mg to 10 mg, from 10 mg to 20 mg, from 10 mg to 15 mg, or from 15 mg to 20 mg. In certain embodiments, the implant comprises from 0.1 mg to 5 mg of the drug (e.g., water-soluble drug, such as pregabalin), such as, for example, from 0.1 mg to 4 mg, from 0.1 mg to 3 mg, from 0.1 mg to 2 mg from 0.1 mg to 1 mg, from 0.5 mg to 5 mg, from 0.5 mg to 4 mg, from 0.5 mg to 3 mg, from 0.5 mg to 2 mg, from 0.5 mg to 1 mg, from 0.5 mg to 1 mg, from 1 mg to 5 mg, from 1 mg to 4 mg, from 1 mg to 3 mg, from 1 mg to 2 mg, from 2 mg to 5 mg, from 2 mg to 4 mg, from 2 mg to 3 mg, from 3 mg to 5 mg, from 3 mg to 4 mg, and from 4 mg to 5 mg. In certain embodiments, the implant comprises from 0.5 mg to 2 mg of the drug (e.g., water-soluble drug, such as pregabalin).

In certain embodiments, the one or more bioresorbable polymers comprise poly(alpha-hydroxy acids), poly(lactide-co-glycolide) (PLG), poly(DL-lactide-co-caprolactone) (DL-PLCL), polycaprolactone (PCL), poly(L-lactic acid) (PLA), poly(trimethylene carbonate) (PTMC), polydioxanone (PDO), poly(4-hydroxy butyrate) (PHB), polyhydroxyalkanoates (PHA), poly(phosphazene), polyphosphate ester, poly(amino acid), polydepsipeptides, poly(butylene succinate) (PBS), polyethylene oxide, polypropylene fumarate, polyiminocarbonates, poly(lactide-co-caprolactone) (PLCL), poly(glycolide-co-caprolactone) (PGCL) copolymer, poly(D,L-lactic acid), polyglycolic acid, poly(L-lactide-co-D,L-lactide), poly(L-lactide-co-glycolide), poly(D,L-lactide-co-glycolide), poly(gycolide-trimethylene carbonate), poly(glycolide-co-caprolactone) (PGCL), poly(ethyl glutamate-co-glutamic acid), poly(tert-butyloxy-carbonylmethyl glutamate), poly(glycerol sebacate), tyrosine-derived polycarbonate, poly 1,3-bis-(p-carboxyphenoxy) hexane-co-sebacic acid, polyphosphazene, ethyl glycinate polyphosphazene, polycaprolactone co-butylacrylate, a copolymer of polyhydroxybutyrate, a copolymer of maleic anhydride, a copolymer of poly(trimethylene carbonate), polyethylene glycol (PEG), hydroxypropylmethylcellulose and cellulose derivatives, polysaccharides (e.g., hyaluronic acid, chitosan and starch), proteins (e.g., gelatin and collagen), polyaspirins, polyphosphagenes, collagen, alginates, albumin, fibrin, vitamin E analogs, D-lactide, D,L-lactide, L-lactide, D,L-lactide-caprolactone (DL-CL), D,L-lactide-glycolide-caprolactone (DL-G-CL), dextrans, vinylpyrrolidone, polyvinyl alcohol (PVA), PVA-g-PLGA, PEGT-PBT copolymer (polyactive), methacrylates, poly(N-isopropylacrylamide), PEO—PPO-PEO (pluronics), PEO—PPO-PAA copolymers, PLGA-PEO-PLGA, PEG-PLG, PLA-PLGA, poloxamer 407, PEG-PLGA-PEG triblock copolymers, SAIB (sucrose acetate isobutyrate) hydroxypropyl cellulose, hydroxypropyl methylcellulose, hydroxyethyl methylcellulose, carboxymethylcellulose or salts thereof, Carbopol®, poly(hydroxyethylmethacrylate), poly(methoxyethylmethacrylate), poly(methoxyethoxy-ethylmethacrylate), polymethylmethacrylate (PMMA), methylmethacrylate (MMA), gelatin, polyvinyl alcohols, propylene glycol, or combinations thereof.

In certain embodiments, the one or more bioresorbable polymers comprise poly(L-lactic acid) (PLA), polycaprolactone (PCL), polyglycolic acid, or a copolymer thereof. In certain embodiments, the one or more bioresorbable polymers comprise poly(L-lactic acid) (PLA). In certain embodiments, the one or more bioresorbable polymers comprise polycaprolactone (PCL). In certain embodiments, the one or more bioresorbable polymers comprise poly(L-lactic acid) (PLA). In certain embodiments, the one or more bioresorbable polymers comprise a copolymer of poly(L-lactic acid) (PLA), polycaprolactone (PCL), and polyglycolic acid.

In certain embodiments, the implant comprises from 50 wt % to 99.9 wt % of the one or more bioresorbable polymers, such as, for example, from 60 wt % to 99.9 wt %, from 70 wt % to 99.9 wt %, from 80 wt % to 99.9 wt %, from 80 wt % to 99.9 wt %, or from 90 wt % to 99.9 wt %. In certain embodiments, the implant comprises from 75 wt % to 99.9 wt % of the one or more bioresorbable polymers, such as, for example, from 75 wt % to 95 wt %, from 80 wt % to 95 wt %, from 85 wt % to 95 wt %, or from 90 wt % to 95 wt %. In certain embodiments, the implant comprises 80 wt % of the one or more bioresorbable polymers.

In certain embodiments, the weight ratio of the drug (e.g., water-soluble drug, such as pregabalin) to one or more bioresorbable polymer is from 1:1 to 1:20, such as, for example, from 1:1 to 1:15, from 1:1 to 1:10, from 1:1 to 1:5, from 1:2 to 1:6, from 1:2 to 1:5, from 1:2 to 1:4, from 1:2 to 1:3, from 1:3 to 1:6, from 1:3 to 1:5, from 1:3 to 1:4, from 1:4 to 1:6, or from 1:4 to 1:5. In certain embodiments, the weight ratio of the drug (e.g., water-soluble drug, such as pregabalin) to one or more bioresorbable polymer is 1:4.

In certain embodiments, the nanoparticles further comprise a permeation enhancer. In certain embodiments, the permeation enhancer is selected from D-α-Tocopherol polyethylene glycol succinate (TPGS), dioctyl sodium sulfosuccinate, sodium caprate, sodium N-[8 (−2-hydroxybenzoyl)amino]caprylate (SNAC), sodium lauryl sulfate, sodium salicylate, oleic acid, lecithin, dehydrated alcohol, polysorbates or polyoxyethylene sorbitan fatty acids esters (Tweens), Sorbitan fatty acids esters (Spans), polyoxyethylene alkyl ether (Brijs), polyoxyl 40 stearate, polyoxy ethylene 50 stearate, polyethylene glycol, polyvinyl alcohol, polyvinylpyrrolidone (e.g., polyvinylpyrrolidone K29-32), hydroxy propyl methyl cellulose, polyvinylpyrrolidone/vinyl acetate (VP/VA) copolymer, poly(lactic-co-glycolic acid), edetate disodium, propylene glycol, glycerol monooleate, bile salts, octoxynol, non-ionic surfactants, anionic surfactants, cationic surfactants, and a combination thereof.

In certain embodiments, when placed in room temperature water for 2 hours the nanoparticles give the water a pH from 5 to 8, such as, for example, a pH from 6 to 8, from 6 to 7, from 7 to 8, or from 6.5 to 7.5.

In certain embodiments, the implant has one of the following shapes: spherical, oblate, ellipsoidal, rod, disc, tube, hemispherical, oval, cylindrical, or irregular. In certain embodiments, the implant is rod-shaped.

In certain embodiments, the implant has the following dimensions: 2 to 10 mm in length and 0.1 mm to 1 mm in diameter, such as, for example, from 3 to 8 mm length and 0.2 mm to 0.8 mm in diameter or 4 mm to 7 mm in length and 0.3 mm to 0.6 mm in diameter. In certain embodiments, the implant is 6 mm in length and 0.5 mm in diameter.

In certain embodiments, the nanoparticles have an average particle size from 300 nm to 1,000 nm in diameter, such as, for example, from 300 nm to 900 nm, from 300 nm to 800 nm, from 300 nm to 700 nm, from 300 nm to 600 nm, from 300 nm to 500 nm, from 300 nm to 400 nm, from 500 nm to 1,000 nm, from 500 nm to 900 nm, from 500 nm to 800 nm, from 500 nm to 700 nm, from 500 nm to 600 nm, from 750 nm to 1,000 nm, from 750 nm to 900 nm, or from 750 nm to 800 nm. In certain embodiments, the nanoparticles are from 300 nm to 400 nm in diameter. In certain other embodiments, the nanoparticles are from 900 nm to 1,000 nm in diameter.

In certain embodiments, the polydispersity index (PDI) of the nanoparticles is from 0.1 to 1, such as, for example, from 0.1 to 0.7, from 0.1 to 0.5, from 0.1 to 0.3, from 0.3 to 0.7, from 0.3 to 0.5, from 0.4 to 0.7, or from 0.4 to 0.6.

In certain embodiments, the implant weighs from 0.5 mg to 10 mg, such as, for example, from 0.5 mg to 7.5 mg from 0.5 mg to 5 mg, from 0.5 mg to 2.5 mg, from 2.5 mg to 10 mg, from 2.5 mg to 7.5 mg, from 2.5 mg to 5 mg, from 5 mg to 10 mg, from 5 mg to 7.5 mg, or from 7.5 mg to 10 mg. In certain embodiments, the implant weighs from 3 mg to 8 mg, from 3 mg to 6 mg or from 4 mg to 6 mg. In certain embodiments, the implant weighs 5 mg.

### 2.3 Polymer Coating

In certain embodiments, the implant further comprises a polymer coating. In certain embodiments, the polymer coating comprises polyethylene glycol, a zwitterionic polymer, a hydrophobic bioresorbable polymer, a bioadhesive polymer, or a combination thereof.

In certain embodiments, the polymer coating comprises a zwitterionic polymer. In certain embodiments, the zwitterionic polymer is selected from poly(sulfobetaine methacrylate), poly(carboxybetaine methacrylate), and a combination thereof.

In certain embodiments, the polymer coating comprises a bioresorbable polymer. In certain embodiments, the polymer coating is the same as the bioresorbable polymer described hereinabove. In certain embodiments, the bioresorbable polymer coating is different than the bioresorbable polymer described hereinabove.

In certain embodiments, the polymer coating comprises a hydrophobic bioresorbable polymer. In certain embodiments, the hydrophobic bioresorbable polymer comprises polycaprolactone (PCL).

In certain embodiments, the polymer coating comprises a bioadhesive polymer. In certain embodiments, the bioadhesive polymer is selected from a polyacrylic acid derivative, cellulose derivative, natural polymer, polyvinyl pyrrolidone (PVP), dextran polymer, polyethylene oxide polymer, thermoreversible polymer, ionic responsive polymer, copolymer of polymethyl vinyl ether and maleic anhydride, and a combination thereof.

In certain embodiments, the polymer coating comprises a bioadhesive, natural polymer. In certain embodiments, the bioadhesive, natural polymer is selected from gum arabic, tragacanth gum, agar polymer, xanthan gum, copolymer of alginic acid and sodium alginate, chitosan polymer, pectin, carrageenan, pullulan polymer, modified starch, and a combination thereof.

In certain embodiments, the polymer coating comprises a biocompatible polymer, e.g., a Eudragrit. Eudragits are, generally, acrylate-methacrylate copolymers, optionally including quaternary ammonium groups, that are commercially available from Rohm Pharma under the tradename Eudragit®. Exemplary Eudragits include those suitable for sustained release, e.g., Eudragit® RL PO, Eudragit® RL 100, Eudragit® RL 30 D, Eudragit® RL 12.5, Eudragit® RS PO, Eudragit® RS 100, Eudragit® RS 30S, and Eudragit® 12.5.

### 2.4 Implant Properties

In certain embodiments, the implant provides sustained release of the drug (e.g., water-soluble drug) for at least six months when measured under Testing Conditions, such as, for example, at least 7 months, at least 8 months, at least 9 months, at least 10 months, at least 11 months, at least 12 months, at least 13 months, at least 14 months, at least 16 months, at least 18 months, or at least 24 months.

In certain embodiments, the implant provides sustained release of drug for 9 months to 24 months, e.g., 9 months to 18 months, 9 months to 14 months, 9 months to 12 months, 12 months to 16 months, or 12 months to 14 months.

The Testing Conditions are placing the implant in the first chamber of a two-chamber 1500 μl fast micro-equilibrium dialyzer (Harvard Apparatus Co., Holliston, MA) fitted with semipermeable regenerated cellulose membranes (Molecular weight cut off 1,000 Da) separating the first and second chambers, wherein the second chamber contains PBS (pH 7.4), maintaining the dialyzer in a thermostatically controlled shaker at 37° C. and 50 rpm, and withdrawing samples from the second chamber for evaluation of drug content by HPLC at predetermined time points.

In certain embodiments, the implant releases no more than 40 wt % of the drug (e.g., water-soluble drug) after six months under Testing Conditions, such as, for example, no more than 50 wt %, no more than 60 wt %, no more than 70 wt %, or no more than 80 wt %. In certain embodiments, the implant releases no more than 60 wt % of the drug after six months under Testing Conditions.

In certain embodiments, the implant releases no more than 80 wt % of the drug (e.g., water-soluble drug) after 11 months under Testing Conditions, such as, for example, no more than 75 wt %, or no more than 70 wt %.

In certain embodiments, the implant releases at least 70 wt % of the drug (e.g., water-soluble drug) after 11 months under Testing Conditions, such as, for example, at least 75 wt % or at least 80 wt %.

In certain embodiments, the implant releases at least 70 wt % of the drug (e.g., water-soluble drug) after 11 months under Testing Conditions, such as, for example, at least 75 wt %, at least 80 wt %, at least 85 wt %, at least 90 wt %, or at least 95 wt %.

In certain embodiments, the implant releases no more than 90 wt % of the drug (e.g., water-soluble drug) after fourteen months under Testing Conditions, such as, for example, no more than 85 wt %, or no more than 80 wt %.

In certain embodiments, the implant releases at least 80 wt % of the drug (e.g., water-soluble drug) after 14 months under Testing Conditions, such as, for example, at least 85 wt % or at least 90 wt %.

In certain embodiments, the implant releases no more than 40 wt % of the drug (e.g., water-soluble drug) after six months, no more than 80 wt % of the drug after 11 months, and no more than 90 wt % of the drug after 14 months under Testing Conditions.

In certain embodiments, the implant releases from 0.5 to 5 μg/day of the drug (e.g., water-soluble drug) under Testing Conditions, such as, for example, from 0.5 μg/day to 4 μg/day, from 0.5 μg/day to 3 μg/day, from 0.5 μg/day to 2 μg/day, from 0.5 μg/day to 1 μg/day, from 1 μg/day to 5 μg/day, from 1 μg/day to 4 μg/day, from 1 μg/day to 3 μg/day, from 1 μg/day to 2 μg/day, from 2 μg/day to 5 μg/day, from 2 μg/day to 4 μg/day, from 3 μg/day to 5 μg/day, from 3 μg/day to 4 μg/day, from 4 μg/day to 5 μg/day. In certain embodiments, the implant releases from 1 to 3 μg/day of the drug under Testing Conditions.

In certain embodiments, the implant has the following release profile under Testing Conditions: (i) provides sustained release of the drug (e.g., water-soluble drug) for at least six months and (ii) releases no more than 40 wt % of the drug after six months.

In certain embodiments, the implant has the following release profile under Testing Conditions: (i) provides sustained release of the drug (e.g., water-soluble drug) for at least six months; (ii) releases no more than 80 wt % of the drug after 11 months; and (iii) releases no more than 90 wt % of the drug after 14 months.

In certain embodiments, the implant has the following release profile under Testing Conditions: (i) provides sustained release of the drug (e.g., water-soluble drug) for at least six months and (ii) releases from 0.5 to 5 μg/day of the drug.

In certain embodiments, the implant has the following release profile under Testing Conditions: (i) provides sustained release of the drug (e.g., water-soluble drug) for at least six months, (ii) releases no more than 40 wt % of the drug after six months, (iii) releases no more than 80 wt % of the drug after 11 months, and (iv) releases no more than 90 wt % of the drug after 14 months.

In certain embodiments, the implant has the following release profile under Testing Conditions: (i) provides sustained release of the drug (e.g., water-soluble drug) for at least six months, (ii) releases no more than 40 wt % of the drug after six months, and (iii) releases from 0.5 to 5 μg/day of the drug.

In certain embodiments, the implant has the following release profile under Testing Conditions: (i) provides sustained release of the drug (e.g., water-soluble drug) for at least six months, (ii) releases no more than 80 wt % of the drug after 11 months, and (iii) releases from 0.5 to 5 μg/day of the drug.

In certain embodiments, the implant has the following release profile under Testing Conditions: (i) provides sustained release of the drug (e.g., water-soluble drug) for at least six months, (ii) releases no more than 40 wt % of the drug after six months, (iii) releases no more than 80 wt % of the after 11 months, and (iv) releases from 0.5 to 5 μg/day of the drug.

In certain embodiments, the implant has the following release profile under Testing Conditions: (i) provides sustained release of the drug (e.g., water-soluble drug) for at least six months, (ii) releases no more than 40 wt % of the drug after six months, (iii) releases no more than 80 wt % of the drug after 11 months, (iv) releases no more than 90 wt % of the drug after 14 months, and (v) releases from 0.5 to 5 μg/day of the drug.

In certain embodiments, the implant provides zero-order release of the drug. In certain other embodiments, the implant provides Higuchi release of the drug, i.e., a rate of drug release that follows the Higuchi model. In certain embodiments, the implant provides Higuchi Fickian drug release. In still other embodiments, the implant provides first order drug release.

In certain embodiments, when placed in water for 2 h, the implant gives the water a pH from 5 to 8, such as, for example, from 5 to 7, from 5 to 6, from 6 to 8, from 6 to 7, or from 7 to 8. In certain embodiments, the pH is selected from 5, 6, 7, or 8.

In certain embodiments, the implant does not swell by more than 50% of the initial implant weight when incubated at 37° C. in a plate containing 2% w/v agar gel for 1 hour, such as, for example, no more than 40% of the initial implant weight, no more than 30% of the initial implant weight, no more than 20% of the initial implant weight, no more than 10% of the initial implant weight, or no more than 5% of the initial implant weight.

In certain embodiments, the implant does not swell by more than 15% of the initial implant weight when incubated at 37° C. in a plate containing 2% w/v agar gel for 1 hour, such as, for example, no more than 10% of the initial implant weight, or no more than 5% of the initial implant weight.

In certain embodiments, the implant does not swell by more than 20% of the initial implant weight when incubated at 37° C. in a plate containing 2% w/v agar gel for 2 hours, such as, for example, no more than 15% of the initial implant weight, no more than 10% of the initial implant weight, or no more than 5% of the initial implant weight.

In certain embodiments, the implant does not swell by more than 25% of the initial implant weight when incubated at 37° C. in a plate containing 2% w/v agar gel for 6 hours, such as, for example, no more than 20% of the initial implant weight, no more than 15% of the initial implant weight, no more than 10% of the initial implant weight, or no more than 5% of the initial implant weight.

In certain embodiments, the implant does not swell by more than 35% of the initial implant weight when incubated at 37° C. in a plate containing 2% w/v agar gel for 8 hours, such as, for example, no more than 30% of the initial implant weight, no more than 25% of the initial implant weight, no more than 20% of the initial implant weight, no more than 15% of the initial implant weight, no more than 10% of the initial implant weight, or no more than 5% of the initial implant weight.

In certain embodiments, the implant does not swell by more than 45% of the initial implant weight when incubated at 37° C. in a plate containing 2% w/v agar gel for 48 hours, such as, for example, no more than 40% of the initial implant weight, no more than 35% of the initial implant weight, no more than 30% of the initial implant weight, no more than 25% of the initial implant weight, no more than 20% of the initial implant weight, no more than 15% of the initial implant weight, no more than 10% of the initial implant weight, or no more than 5% of the initial implant weight.

In certain embodiments, the implant does not swell by more than 60% of the initial implant weight when incubated at 37° C. in a plate containing 2% w/v agar gel for 72 hours, such as, for example, no more than 55% of the initial implant weight, no more than 50% of the initial implant weight, no more than 45% of the initial implant weight, no more than 40% of the initial implant weight, no more than 35% of the initial implant weight, no more than 30% of the initial implant weight, no more than 25% of the initial implant weight, no more than 20% of the initial implant weight, no more than 15% of the initial implant weight, no more than 10% of the initial implant weight, or no more than 5% of the initial implant weight.

In certain embodiments, when the implant is incubated at 37° C. in a plate containing 2% w/v agar gel, at least two of the following are true:
(i) the implant does not swell by more than 15% of the initial implant weight after 1 hour;
(ii) the implant does not swell by more than 20% of the initial implant weight after 2 hours;
(iii) the implant does not swell by more than 25% of the initial implant weight after 6 hours;
(iv) the implant does not swell by more than 35% of the initial implant weight after 8 hours;
(v) the implant does not swell by more than 45% of the initial implant wight after 48 hours; and
(vi) the implant does not swell by more than 60% of the initial implant weight after 72 hours.

In some embodiments, the implant is a nanofiber implant. In some embodiments, the implant is a nanoparticle implant.

In certain embodiments, the nanofiber implant does not swell by more than 15% of the initial implant weight when incubated at 37° C. in a plate containing 2% w/v agar gel for 1 hour, such as, for example, no more than 10% of the initial implant weight, or no more than 5% of the initial implant weight.

In certain embodiments, the nanofiber implant does not swell by more than 20% of the initial implant weight when incubated at 37° C. in a plate containing 2% w/v agar gel for 2 hours, such as, for example, no more than 15% of the initial implant weight, no more than 10% of the initial implant weight, or no more than 5% of the initial implant weight.

In certain embodiments, the nanofiber implant does not swell by more than 25% of the initial implant weight when incubated at 37° C. in a plate containing 2% w/v agar gel for 6 hours, such as, for example, no more than 20% of the initial implant weight, no more than 15% of the initial implant weight, no more than 10% of the initial implant weight, or no more than 5% of the initial implant weight.

In certain embodiments, the nanofiber implant does not swell by more than 35% of the initial implant weight when incubated at 37° C. in a plate containing 2% w/v agar gel for 8 hours, such as, for example, no more than 30% of the initial implant weight, no more than 25% of the initial implant weight, no more than 20% of the initial implant weight, no more than 15% of the initial implant weight, no more than 10% of the initial implant weight, or no more than 5% of the initial implant weight.

In certain embodiments, the nanofiber implant does not swell by more than 45% of the initial implant weight when incubated at 37° C. in a plate containing 2% w/v agar gel for 48 hours, such as, for example, no more than 40% of the initial implant weight, no more than 35% of the initial implant weight, no more than 30% of the initial implant weight, no more than 25% of the initial implant weight, no more than 20% of the initial implant weight, no more than 15% of the initial implant weight, no more than 10% of the initial implant weight, or no more than 5% of the initial implant weight.

In certain embodiments, the nanofiber implant does not swell by more than 60% of the initial implant weight when incubated at 37° C. in a plate containing 2% w/v agar gel for 72 hours, such as, for example, no more than 55% of the initial implant weight, no more than 50% of the initial implant weight, no more than 45% of the initial implant weight, no more than 40% of the initial implant weight, no more than 35% of the initial implant weight, no more than 30% of the initial implant weight, no more than 25% of the initial implant weight, no more than 20% of the initial implant weight, no more than 15% of the initial implant weight, no more than 10% of the initial implant weight, or no more than 5% of the initial implant weight.

In certain embodiments, when the nanofiber implant is incubated at 37° C. in a plate containing 2% w/v agar gel, at least two of the following are true:
(i) the nanofiber implant does not swell by more than 15% of the initial implant weight after 1 hour;
(ii) the nanofiber implant does not swell by more than 20% of the initial implant weight after 2 hours;
(iii) the nanofiber implant does not swell by more than 25% of the initial implant weight after 6 hours;
(iv) the nanofiber implant does not swell by more than 35% of the initial implant weight after 8 hours;
(v) the nanofiber implant does not swell by more than 45% of the initial implant wight after 48 hours; and
(vi) the nanofiber implant does not swell by more than 60% of the initial implant weight after 72 hours.

In certain embodiments, the nanoparticle implant does not swell by more than 25% of the initial implant weight when incubated at 37° C. in a plate containing 2% w/v agar gel for 1 hour, such as, for example, no more than 20% of the initial implant weight, no more than 15% of the initial implant weight, no more than 10% of the initial implant weight, or no more than 5% of the initial implant weight.

In certain embodiments, the nanoparticle implant does not swell by more than 35% of the initial implant weight when incubated at 37° C. in a plate containing 2% w/v agar gel for 2 hours, such as, for example, no more than 30% of the initial implant weight, no more than 25% of the initial implant weight, no more than 20% of the initial implant weight, no more than 15% of the initial implant weight, no more than 10% of the initial implant weight, or no more than 5% of the initial implant weight.

In certain embodiments, the nanoparticle implant does not swell by more than 40% of the initial implant weight when incubated at 37° C. in a plate containing 2% w/v agar gel for 6 hours, such as, for example, no more than 35% of the initial implant weight, no more than 30% of the initial implant weight, no more than 25% of the initial implant weight, no more than 20% of the initial implant weight, no more than 15% of the initial implant weight, no more than 10% of the initial implant weight, or no more than 5% of the initial implant weight.

In certain embodiments, the nanoparticle implant does not swell by more than 45% of the initial implant weight when incubated at 37° C. in a plate containing 2% w/v agar gel for 24 hours, such as, for example, no more than 40% of the initial implant weight, no more than 35% of the initial implant weight, no more than 30% of the initial implant weight, no more than 25% of the initial implant weight, no more than 20% of the initial implant weight, no more than 15% of the initial implant weight, no more than 10% of the initial implant weight, or no more than 5% of the initial implant weight.

In certain embodiments, the nanoparticle implant does not swell by more than 50% of the initial implant weight when incubated at 37° C. in a plate containing 2% w/v agar gel for 48 hours, such as, for example, no more than 45% of the initial implant weight, no more than 40% of the initial implant weight, no more than 35% of the initial implant weight, no more than 30% of the initial implant weight, no more than 25% of the initial implant weight, no more than 20% of the initial implant weight, no more than 15% of the initial implant weight, no more than 10% of the initial implant weight, or no more than 5% of the initial implant weight.

In certain embodiments, the nanoparticle implant does not swell by more than 60% of the initial implant weight when incubated at 37° C. in a plate containing 2% w/v agar gel for 72 hours, such as, for example, no more than 55% of the initial implant weight, no more than 50% of the initial implant weight, no more than 45% of the initial implant weight, no more than 40% of the initial implant weight, no more than 35% of the initial implant weight, no more than 30% of the initial implant weight, no more than 25% of the initial implant weight, no more than 20% of the initial implant weight, no more than 15% of the initial implant weight, no more than 10% of the initial implant weight, or no more than 5% of the initial implant weight.

In certain embodiments, when the nanoparticle implant is incubated at 37° C. in a plate containing 2% w/v agar gel, at least two of the following are true:
(i) the nanoparticle implant does not swell by more than 25% of the initial implant weight after 1 hour;
(ii) the nanoparticle implant does not swell by more than 35% of the initial implant weight after 2 hours;
(iii) the nanoparticle implant does not swell by more than 40% of the initial implant weight after 6 hours;
(iv) the nanoparticle implant does not swell by more than 45% of the initial implant weight after 8 hours;
(v) the nanoparticle implant does not swell by more than 50% of the initial implant wight after 48 hours; and
(vi) the nanoparticle implant does not swell by more than 60% of the initial implant weight after 72 hours.

In certain embodiments, the implant exhibits 50 wt % or less moisture uptake when incubated in a desiccator with a saturated aqueous solution of ammonium chloride (relative humidity 79.5%) for 3 days, such as, for example, 40 wt % or less, 30 wt % or less, 20 wt % or less, 10 wt % or less, or 5 wt % or less.

In certain embodiments, the implant exhibits 10 wt % or less moisture uptake when incubated in a desiccator with a saturated aqueous solution of ammonium chloride (relative humidity 79.5%) for 3 days, such as, for example, 9 wt % or less, 8 wt % or less, 7 wt % or less, 6 wt % or less, 5 wt % or less, 4 wt % or less, 3 wt % or less, 2 wt % or less, or 1 wt % or less.

In certain embodiments, the implant exhibits 5 wt % or less moisture uptake when incubated in a desiccator with a saturated aqueous solution of ammonium chloride (relative humidity 79.5%) for 3 days, such as, for example, 4 wt % or less, 3 wt % or less, 2 wt % or less, or 1 wt % or less.

In certain embodiments, the implant exhibits 3 wt % or less moisture uptake when incubated in a desiccator with a saturated aqueous solution of ammonium chloride (relative humidity 79.5%) for 3 days, such as, for example, 2 wt % or less, or 1 wt % or less.

In certain embodiments, the implant exhibits less fouling than the control implant when evaluated under the following conditions:
Plastic sheets (polypropylene, PP) are cut into the same shape and dimensions of the implants (rod shape) and used as a control. The implant as well as the PP rods are placed in 24-well plate and incubated with 1 ml of fibrinogen solution (10 mg/ml) in PBS for 2 h. After 2 h, the implants and the PP rods are washed 5 times with PBS then transferred to a new 24-well plate and incubated with 1 ml of horseradish peroxidase-conjugated antifibrinogen (1 μg/ml) in PBS for 1h, followed by 5 washes with PBS. Implants and PP rods are transferred to a new 24-well plate and incubated for 15 min with 1 ml of o-phenylenediamine citrate phosphate solution [1 mg/ml; 0.1M (pH 5)] containing 0.03% hydrogen peroxide. After 15 min, the enzymatic activity is stopped by addition 1 ml of 1M HCl. The plate absorbance at 492 nm is measured using a plate reader spectrophotometer. The absorbance of the wells containing the implants will be calculated as a percentage from the absorbance of the wells containing the PP rods.

In certain embodiments, the implant described herein exhibits at least 10% less protein fouling compared to the control implant, such as, for example, at least 20% less protein fouling, at least 30% less protein fouling, at least 40% protein fouling, at least 50% less protein fouling, at least 60% less protein fouling, at least 70% less protein fouling, at least 80% less protein fouling, or at least 90% less protein fouling.

In certain embodiments, the implant exhibits an ex vivo trans-scleral rate of drug permeation from 0.001 to 1 μg/min, such as, for example, 0.01 to 1 μg/min, 0.05 to 1 μg/min, 0.1 to 1 μg/min, 0.2 to 1 μg/min, 0.3 to 1 μg/min, 0.4 to 1 μg/min, 0.5 to 1 μg/min, 0.6 to 1 μg/min, 0.7 to 1 μg/min, 0.8 to 1 μg/min, 0.9 to 1 μg/min, 0.01 to 0.9 μg/min, 0.05 to 0.9 μg/min, 0.1 to 0.9 μg/min, 0.2 to 0.9 μg/min, 0.3 to 0.9 μg/min, 0.4 to 0.9 μg/min, 0.5 to 0.9 μg/min, 0.6 to 0.9 μg/min, 0.7 to 0.9 μg/min, 0.8 to 0.9 μg/min, 0.01 to 0.8 g/min, 0.05 to 0.8 μg/min, 0.1 to 0.8 μg/min, 0.2 to 0.8 μg/min, 0.3 to 0.8 μg/min, 0.4 to 0.8 μg/min, 0.5 to 0.8 μg/min, 0.6 to 0.8 μg/min, 0.7 to 0.8 μg/min, 0.01 to 0.7 μg/min, 0.05 to 0.7 μg/min, 0.1 to 0.7 μg/min, 0.2 to 0.7 μg/min, 0.3 to 0.7 μg/min, 0.4 to 0.7 μg/min, 0.5 to 0.7 μg/min, 0.6 to 0.7 μg/min, 0.01 to 0.6 μg/min, 0.05 to 0.6 μg/min, 0.1 to 0.6 μg/min, 0.2 to 0.6 μg/min, 0.3 to 0.6 μg/min, 0.4 to 0.6 μg/min, 0.5 to 0.6 μg/min, 0.01 to 0.5 μg/min, 0.05 to 0.5 μg/min, 0.1 to 0.5 μg/min, 0.2 to 0.5 μg/min, 0.3 to 0.5 μg/min, 0.4 to 0.5 μg/min, 0.01 to 0.4 μg/min, 0.05 to 0.4 μg/min, 0.1 to 0.4 μg/min, 0.2 to 0.4 μg/min, 0.3 to 0.4 μg/min, 0.01 to 0.3 μg/min, 0.05 to 0.3 μg/min, 0.1 to 0.3 μg/min, 0.2 to 0.3 μg/min, 0.01 to 0.2 μg/min, 0.05 to 0.2 μg/min, 0.1 to 0.2 μg/min, 0.01 to 0.1 μg/min, 0.05 to 0.1 μg/min, or 0.01 to 0.01 μg/min.

In certain embodiments, the implant exhibits an ex vivo trans-scleral rate of drug permeation from 0.1 to 1.5 μg/min, such as, for example, 0.2 to 1.5 μg/min, 0.3 to 1.5 μg/min, 0.4 to 1.5 μg/min, 0.5 to 1.5 μg/min, 0.6 to 1.5 g/min, 0.7 to 1.5 μg/min, 0.8 to 1.5 μg/min, 0.9 to 1.5 μg/min, 1.0 to 1.5 μg/min, 1.1 to 1.5 μg/min, 1.2 to 1.5 μg/min, 1.3 to 1.5 μg/min, 1.4 to 1.5 μg/min, 0.1 to 1.4 μg/min, 0.2 to 1.4 μg/min, 0.3 to 1.4 μg/min, 0.4 to 1.4 μg/min, 0.5 to 1.4 μg/min, 0.6 to 1.4 μg/min, 0.7 to 1.4 μg/min, 0.8 to 1.4 μg/min, 0.9 to 1.4 μg/min, 1.0 to 1.4 μg/min, 1.1 to 1.4 μg/min, 1.2 to 1.4 μg/min, 1.3 to 1.4 μg/min, 0.1 to 1.3 μg/min, 0.2 to 1.3 μg/min, 0.3 to 1.3 μg/min, 0.4 to 1.3 μg/min, 0.5 to 1.3 μg/min, 0.6 to 1.3 μg/min, 0.7 to 1.3 μg/min, 0.8 to 1.3 μg/min, 0.9 to 1.3 μg/min, 1.0 to 1.3 μg/min, 1.1 to 1.3 μg/min, 1.2 to 1.3 μg/min, 0.1 to 1.2 μg/min, 0.2 to 1.2 μg/min, 0.3 to 1.2 μg/min, 0.4 to 1.2 μg/min, 0.5 to 1.2 μg/min, 0.6 to 1.2 μg/min, 0.7 to 1.2 μg/min, 0.8 to 1.2 μg/min, 0.9 to 1.2 μg/min, 1.0 to 1.2 μg/min, 1.1 to 1.2 μg/min, 0.1 to 1.1 μg/min, 0.2 to 1.1 μg/min, 0.3 to 1.1 μg/min, 0.4 to 1.1 μg/min, 0.5 to 1.1 μg/min, 0.6 to 1.1 μg/min, 0.7 to 1.1 μg/min, 0.8 to 1.1 μg/min, 0.9 to 1.1 μg/min, 1.0 to 1.1 μg/min, 0.1 to 1.0 μg/min, 0.2 to 1.0 μg/min, 0.3 to 1.0 μg/min, 0.4 to 1.0 μg/min, 0.5 to 1.0 μg/min, 0.6 to 1.0 μg/min, 0.7 to 1.0 g/min, 0.8 to 1.0 μg/min, 0.9 to 1.0 μg/min, 0.1 to 0.9 μg/min, 0.2 to 0.9 μg/min, 0.3 to 0.9 μg/min, 0.4 to 0.9 μg/min, 0.5 to 0.9 μg/min, 0.6 to 0.9 μg/min, 0.7 to 0.9 μg/min, 0.8 to 0.9 μg/min, 0.1 to 0.8 μg/min, 0.2 to 0.8 μg/min, 0.3 to 0.8 μg/min, 0.4 to 0.8 μg/min, 0.5 to 0.8 μg/min, 0.6 to 0.8 μg/min, 0.7 to 0.8 μg/min, 0.1 to 0.7 μg/min, 0.2 to 0.7 μg/min, 0.3 to 0.7 μg/min, 0.4 to 0.7 μg/min, 0.5 to 0.7 μg/min, 0.6 to 0.7 μg/min, 0.1 to 0.6 μg/min, 0.2 to 0.6 μg/min, 0.3 to 0.6 μg/min, 0.4 to 0.6 μg/min, 0.5 to 0.6 μg/min, 0.1 to 0.5 μg/min, 0.2 to 0.5 μg/min, 0.3 to 0.5 μg/min, 0.4 to 0.5 μg/min, 0.1 to 0.4 μg/min, 0.2 to 0.4 μg/min, 0.3 to 0.4 μg/min, 0.1 to 0.3 μg/min, 0.2 to 0.3 μg/min, and 0.1 to 0.2 μg/min.

In certain embodiments, the implant exhibits an ex vivo trans-scleral rate of drug permeation from 0.001 to 1 μg/min, such as, for example, 0.01 to 1 μg/min, 0.05 to 1 μg/min, 0.1 to 1 μg/min, 0.2 to 1 μg/min, 0.3 to 1 μg/min, 0.4 to 1 μg/min, 0.5 to 1 μg/min, 0.6 to 1 μg/min, 0.7 to 1 μg/min, 0.8 to 1 μg/min, 0.9 to 1 μg/min, 0.01 to 0.9 μg/min, 0.05 to 0.9 μg/min, 0.1 to 0.9 μg/min, 0.2 to 0.9 μg/min, 0.3 to 0.9 μg/min, 0.4 to 0.9 μg/min, 0.5 to 0.9 μg/min, 0.6 to 0.9 μg/min, 0.7 to 0.9 μg/min, 0.8 to 0.9 μg/min, 0.01 to 0.8 μg/min, 0.05 to 0.8 μg/min, 0.1 to 0.8 μg/min, 0.2 to 0.8 μg/min, 0.3 to 0.8 μg/min, 0.4 to 0.8 μg/min, 0.5 to 0.8 μg/min, 0.6 to 0.8 μg/min, 0.7 to 0.8 μg/min, 0.01 to 0.7 μg/min, 0.05 to 0.7 μg/min, 0.1 to 0.7 μg/min, 0.2 to 0.7 μg/min, 0.3 to 0.7 μg/min, 0.4 to 0.7 μg/min, 0.5 to 0.7 μg/min, 0.6 to 0.7 μg/min, 0.01 to 0.6 μg/min, 0.05 to 0.6 μg/min, 0.1 to 0.6 μg/min, 0.2 to 0.6 μg/min, 0.3 to 0.6 μg/min, 0.4 to 0.6 μg/min, 0.5 to 0.6 μg/min, 0.01 to 0.5 μg/min, 0.05 to 0.5 μg/min, 0.1 to 0.5 μg/min, 0.2 to 0.5 μg/min, 0.3 to 0.5 μg/min, 0.4 to 0.5 μg/min, 0.01 to 0.4 μg/min, 0.05 to 0.4 μg/min, 0.1 to 0.4 μg/min, 0.2 to 0.4 μg/min, 0.3 to 0.4 μg/min, 0.01 to 0.3 μg/min, 0.05 to 0.3 μg/min, 0.1 to 0.3 μg/min, 0.2 to 0.3 μg/min, 0.01 to 0.2 μg/min, 0.05 to 0.2 μg/min, 0.1 to 0.2 μg/min, 0.01 to 0.1 μg/min, 0.05 to 0.1 μg/min, or 0.01 to 0.01 μg/min.

In certain embodiments, the implant exhibits an ex vivo trans-scleral average drug flux from 0.0005 to 5 μg/cm²·hr. In certain embodiments, the implant provides an ex vivo trans-scleral average drug flux from 0.0005 to 1 μg/cm²·hr. In certain embodiments, the implant provides an ex vivo trans-scleral average drug flux from 0.0005 to 0.1 μg/cm²·hr. In certain embodiments, the implant provides an ex vivo trans-scleral average drug flux from 0.0005 to 0.05 μg/cm²·hr, such as, for example, 0.0006 to 0.05 μg/cm²·hr, 0.0007 to 0.05 μg/cm²·hr, 0.0008 to 0.05 μg/cm²·hr, 0.0009 to 0.05 μg/cm²·hr, 0.001 to 0.05 μg/cm²·hr, 0.002 to 0.05 μg/cm²·hr, 0.003 to 0.05 μg/cm²·hr, 0.004 to 0.05 μg/cm²·hr, 0.005 to 0.05 μg/cm²·hr, 0.006 to 0.05 μg/cm²·hr, 0.007 to 0.05 μg/cm²·hr, 0.008 to 0.05 μg/cm²·hr, 0.009 to 0.05 μg/cm²·hr, 0.01 to 0.05 μg/cm²·hr, 0.02 to 0.05 μg/cm²·hr, 0.03 to 0.05 μg/cm²·hr, 0.04 to 0.05 μg/cm²·hr, 0.0005 to 0.04 μg/cm²·hr, 0.0006 to 0.04 μg/cm²·hr, 0.0007 to 0.04 g/cm²·hr, 0.0008 to 0.04 μg/cm²·hr, 0.0009 to 0.04 μg/cm²·hr, 0.001 to 0.04 μg/cm²·hr, 0.002 to 0.04 μg/cm²·hr, 0.003 to 0.04 μg/cm²·hr, 0.004 to 0.04 g/cm²·hr, 0.005 to 0.04 μg/cm²·hr, 0.006 to 0.04 μg/cm²·hr, 0.007 to 0.04 μg/cm²·hr, 0.008 to 0.04 μg/cm²·hr, 0.009 to 0.04 μg/cm²·hr, 0.01 to 0.04 μg/cm²·hr, 0.02 to 0.04 μg/cm²·hr, 0.03 to 0.04 μg/cm²·hr, 0.0005 to 0.03 μg/cm²·hr, 0.0006 to 0.03 g/cm²·hr, 0.0007 to 0.03 μg/cm²·hr, 0.0008 to 0.03 μg/cm²·hr, 0.0009 to 0.03 μg/cm²·hr, 0.001 to 0.03 μg/cm²·hr, 0.002 to 0.03 μg/cm²·hr, 0.003 to 0.03 μg/cm²·hr, 0.004 to 0.03 μg/cm²·hr, 0.005 to 0.03 μg/cm²·hr, 0.006 to 0.03 μg/cm²·hr, 0.007 to 0.03 μg/cm²·hr, 0.008 to 0.03 μg/cm²·hr, 0.009 to 0.03 μg/cm²·hr, 0.01 to 0.03 μg/cm²·hr, 0.02 to 0.03 μg/cm²·hr, 0.0005 to 0.02 g/cm²·hr, 0.0006 to 0.02 μg/cm²·hr, 0.0007 to 0.02 μg/cm²·hr, 0.0008 to 0.02 μg/cm²·hr, 0.0009 to 0.02 μg/cm²·hr, 0.001 to 0.02 μg/cm²·hr, 0.0005 to 0.01 μg/cm²·hr, 0.0006 to 0.01 μg/cm²·hr, 0.0007 to 0.01 μg/cm²·hr, 0.0008 to 0.01 μg/cm²·hr, 0.0009 to 0.01 μg/cm²·hr, 0.01 to 0.02 μg/cm²·hr, 0.005 to 0.009 μg/cm²·hr, 0.006 to 0.009 μg/cm²·hr, 0.007 to 0.009 μg/cm²·hr, 0.008 to 0.009 g/cm²·hr, 0.005 to 0.008 μg/cm²·hr, 0.006 to 0.008 μg/cm²·hr, 0.007 to 0.008 μg/cm²·hr, 0.005 to 0.007 μg/cm²·hr, 0.006 to 0.007 μg/cm²·hr, or 0.005 to 0.006 μg/cm²·hr, 0.0005 to 0.0009 μg/cm²·hr, 0.0006 to 0.0009 g/cm²·hr, 0.0007 to 0.0009 μg/cm²·hr, 0.0008 to 0.0009 μg/cm²·hr, 0.0005 to 0.0008 μg/cm²·hr, 0.0006 to 0.0008 g/cm²·hr, 0.0007 to 0.0008 μg/cm²·hr, 0.0005 to 0.0007 μg/cm²·hr, 0.0006 to 0.00007 g/cm²·hr, or 0.0005 to 0.006 g/cm²·hr.

In certain embodiments, implant exhibits a permeability coefficient from 0.01·10⁻⁴ to 1·10⁻⁴ cm/min, preferably, from 0.01·10⁻⁴ to 0.5·10⁻⁴ cm/min, more preferably from 0.01·10⁻⁴ to 0.1·10⁻⁴ cm/min, such as, for example, 0.02·10⁻⁴ to 0.1·10⁻⁴ cm/min, 0.03·10⁻⁴ to 0.1·10⁻⁴ cm/min, 0.04·10⁻⁴ to 0.1·10-4 cm/min, 0.05·10⁻⁴ to 0.1·10⁻⁴ cm/min, 0.06·10⁻⁴ to 0.1·10⁻⁴ cm/min, 0.07·10⁻⁴ to 0.1·10⁻⁴ cm/min, 0.08·10⁻⁴ to 0.1·10⁻⁴ cm/min, 0.09·10⁻⁴ to 0.1·10⁻⁴ cm/min, 0.01·10⁻⁴ to 0.09·10⁻⁴ cm/min, 0.02·10⁻⁴ to 0.09·10⁻⁴ cm/min, 0.03·10⁻⁴ to 0.09·10⁻⁴ cm/min, 0.04·10⁻⁴ to 0.09·10⁻⁴ cm/min, 0.05·10⁻⁴ to 0.09·10⁻⁴ cm/min, 0.06·10⁻⁴ to 0.09·10⁻⁴ cm/min, 0.07·10⁻⁴ to 0.09· 10⁻⁴ cm/min, 0.08·10⁻⁴ to 0.09·10⁻⁴ cm/min, 0.01·10-4 to 0.08·10⁻⁴ cm/min, 0.02·10⁻⁴ to 0.08·10⁻⁴ cm/min, 0.03·10⁻⁴ to 0.08·10⁻⁴ cm/min, 0.04·10⁻⁴ to 0.08·10⁻⁴ cm/min, 0.05·10⁻⁴ to 0.08·10⁻⁴ cm/min, 0.06·10⁻⁴ to 0.08·10⁻⁴ cm/min, 0.07·10⁻⁴ to 0.08·10⁻⁴ cm/min, 0.01·10⁻⁴ to 0.07·10⁻⁴ cm/min, 0.02·10⁻⁴ to 0.07·10⁻⁴ cm/min, 0.03·10⁻⁴ to 0.07·10⁻⁴ cm/min, 0.04·10⁻⁴ to 0.07·10⁻⁴ cm/min, 0.05·10⁻⁴ to 0.07·10⁻⁴ cm/min, 0.06·10⁻⁴ to 0.07·10⁻⁴ cm/min, 0.01·10⁻⁴ to 0.06·10⁻⁴ cm/min, 0.02·10⁻⁴ to 0.06·10⁻⁴ cm/min, 0.03·10⁻⁴ to 0.06·10⁻⁴ cm/min, 0.04·10⁻⁴ to 0.06·10⁻⁴ cm/min, 0.05·10⁻⁴ to 0.06·10⁻⁴ cm/min, 0.01·10⁻⁴ to 0.05·10⁻⁴ cm/min, 0.02·10⁻⁴ to 0.05·10⁻⁴ cm/min, 0.03·10⁻⁴ to 0.05·10⁻⁴ cm/min, 0.04·10⁻⁴ to 0.05·10⁻⁴ cm/min, 0.01·10⁻⁴ to 0.04·10⁻⁴ cm/min, 0.02·10⁻⁴ to 0.04·10⁻⁴ cm/min, 0.03·10⁻⁴ to 0.04·10⁻⁴ cm/min, 0.01·10⁻⁴ to 0.03·10⁻⁴ cm/min, 0.02·10⁻⁴ to 0.03·10⁻⁴ cm/min, 0.01·10⁻⁴ to 0.02·10⁻⁴ cm/min, 0.01·10⁻⁴ to 0.001 cm/min, 0.02·10⁻⁴ to 0.001 cm/min, 0.03·10⁻⁴ to 0.001 cm/min, 0.04·10⁻⁴ to 0.001 cm/min, 0.05·10⁻⁴ to 0.001 cm/min, 0.06·10⁻⁴ to 0.001 cm/min, 0.07·10⁻⁴ to 0.001 cm/min, 0.08·10⁻⁴ to 0.001 cm/min, 0.09·10⁻⁴ to 0.001 cm/min, 0.01·10⁻⁴ to 0.09·10⁻⁴ cm/min, 0.01·10⁻⁴ to 0.05·10⁻⁴ cm/min, or 0.01·10⁻⁴ to 0.02·10⁻⁴ cm/min.

In certain embodiments, the implant is stable for at least six months when evaluated under one or more of the following conditions: 25° C. and 60% relative humidity; 30° C. and 65% relative humidity, and 40° C. and 75% relative humidity, such as, for example, at least seven months, at least eight months, at least nine months, at least ten months, at least eleven months, or at least twelve months. “Stable,” as used herein means little to no change in physical appearance (e.g., size, shape, and color), maintaining a pH within the limits tolerated by the eye (3.5-8.5) and having from 90-110% initial drug content when measured by HPLC.

### 3. METHODS OF PREPARING OCULAR IMPLANTS

In one aspect, the present disclosure provides methods of preparing the ocular implants described herein.

In certain embodiments, a method of making an ocular implant comprising a plurality of nanofibers comprises:
dissolving a drug (e.g., water-soluble drug or water-insoluble drug) and one or more bioresorbable polymers in a solvent to provide a solution,
electrospinning the solution to provide a nanofiber sheet comprising a plurality of nanofibers on a collector surface,
collecting the nanofiber sheet from the collector surface, and
cutting the nanofiber sheet to form a plurality of implants, each implant comprising a plurality of nanofibers; wherein
the nanofibers provide for sustained release of the drug from the implant; and
the implant is suitable for implantation into the eye of a mammal.

In embodiments of the methods described herein, the drug is as described hereinabove. In certain embodiments, the drug is pregabalin.

The bioresorbable polymers described hereinabove can be used in the method provided. In certain embodiments, the one or more bioresorbable polymers comprise poly(L-lactic acid) (PLA), polycaprolactone (PCL), polyglycolic acid, or a copolymer thereof. In certain embodiments, the one or more bioresorbable polymers comprise poly(L-lactic acid) (PLA). In certain embodiments, the one or more bioresorbable polymers comprise polycaprolactone (PCL). In certain embodiments, the one or more bioresorbable polymers comprise poly(L-lactic acid) (PLA). In certain embodiments, the one or more bioresorbable polymers comprise a copolymer of poly(L-lactic acid) (PLA), polycaprolactone (PCL), and polyglycolic acid.

The drug and one or more bioresorbable polymers are dissolved any solvent in which both components are soluble. Exemplary solvents include organic solvents, organic acid solvents, inorganic acid solvents, and water.

In certain embodiments, the solvent comprises an organic solvent. In certain embodiments, the organic solvent comprises chloroform, acetone, dichloromethane, dimethylformamide, ethyl acetate, tetrahydrofuran, dimethyl sulfoxide, alcohol (e.g., methanol, ethanol, propanol, isopropanol, trifluoroethanol, hexafluoroisopropanol), or a combination thereof.

In certain embodiments, the solvent comprises an organic acid solvent. In certain embodiments, the organic acid solvent comprises acetic acid, trifluoroacetic acid, propionic acid, or a combination thereof. In certain embodiments, the organic acid solvent comprises acetic acid. In certain embodiments, the organic acid solvent comprises glacial acetic acid. In certain embodiments, the organic acid solvent consists of glacial acetic acid.

In certain embodiments, the solvent comprises less than 5 wt % water, such as, for example, from 0 wt % to 5 wt %, from 0 wt % to 3 wt %, from 0 wt % to 1 wt %, from 0.1 wt % to 5 wt %, from 0.1 wt % to 3 wt %, from 0.1 wt % to 1 wt %, from 0.5 wt % to 5 wt %, from 0.5 wt % to 3 wt %, or from 0.5 wt % to 1 wt %.

In certain embodiments, the drug (e.g., water-soluble drug) and bioresorbable polymer are combined in a weight ratio of 1:1 to 1:20 in the solution, such as, for example, from 1:1 to 1:15, from 1:1 to 1:10, from 1:1 to 1:5, from 1:2 to 1:6, from 1:2 to 1:5, from 1:2 to 1:4, from 1:2 to 1:3, from 1:3 to 1:6, from 1:3 to 1:5, from 1:3 to 1:4, from 1:4 to 1:6, or from 1:4 to 1:5. In certain embodiments, the weight ratio of drug to one or more bioresorbable polymer is 1:4.

In certain embodiments, dissolving a drug (e.g., water-soluble drug) and one or more bioresorbable polymers in a solvent to provide a solution is conducted by heating a mixture of the drug and bioresorbable polymer to promote dissolution. In certain embodiments, the mixture is heated to 30° C. to 100° C., such as, for example, from 30° C. to 75° C., from 30° C. to 50° C., from 50° C. to 100° C., from 50° C. to 75° C., or from 75° C. to 100° C.

In certain embodiments, the drug (e.g., water-soluble drug) and bioresorbable polymer are mixed, e.g., shaken or stirred, to promote dissolution.

Once the solution of the drug and one or more bioresorbable polymers is prepared, the solution is electrospun using an electrospinner, e.g., a Spinbox from NanoScience Instruments (Phoenix, AZ), to provide a nanofiber sheet comprising a plurality of nanofibers deposited on a collector surface (e.g., the collector surface of the electrospinner). A person of skill in the art will understand that the parameters of the electrospinner will vary depending on the components being electrospun and their relative amounts.

In certain embodiments, the electrospinner has one spray needle. In other embodiments, the electrospinner has multiple spray needles, e.g., two or more, three or more, or four or more spray needles.

In certain embodiments, coaxial electrospinning is used. In coaxial electrospinning, the main modification relies on the spinneret, which consists of two capillary tubes where a smaller one is inserted (inner) concentrically inside the bigger (outer) capillary to make co-axial configuration.

In some embodiments, the solution is electrospun at a flow rate of 1 μl/min to 20 μl/min, such as, for example, from 1 μl/min to 15 μl/min, from 1 μl/min to 10 μl/min, from 5 μl/min to 20 μl/min, from 5 μl/min to 15 μl/min, from 5 μl/min to 10 μl/min, from 10 μl/min to 20 μl/min, from 10 μl/min to 15 μl/min, or from 15 μl/min to 20 μl/min. In certain embodiments, the solution is electrospun at a flow rate of 8 μl/min.

In certain embodiments, the distance between the tip of the spray nozzle of the electrospinner and the collector surface is from 5 cm to 20 cm, such as, for example, from 5 cm to 15 cm, from 5 cm to 10 cm, from 10 cm to 20 cm, from 10 cm to 15 cm, from 15 cm to 20 cm, or from 13 cm to 19 cm.

In certain embodiments, the applied potential difference between the spray nozzle of the electrospinner and the collector surface is 10 kV to 22 kV, such as, for example, from 10 kV to 20 kV, from 10 kV to 15 kV, from 15 kV to 22 kV, or from 15 kV to 20 kV. In certain embodiments, the applied potential difference is 17 kV.

In certain embodiments, the nanofiber sheet has thickness greater than 2 mm, such as, for example, from 2 mm to 10 mm, from 2 mm to 8 mm, or from 2 mm to 5 mm.

In certain embodiments, the nanofiber sheet is collected from the collector surface in one piece. In certain embodiments, the nanofiber sheet is collected from the surface in multiple pieces.

In certain embodiments, the nanofiber sheet is cut into a plurality of implants using a cutting punch. In certain embodiments, the nanofiber sheet is cut into a plurality of implants using an oval-hole punch.

In certain embodiments, the nanofiber sheet is cut to provide implants having one or more of the following shapes: spherical, oblate, ellipsoidal, rod, disc, tube, hemispherical, oval, cylindrical or irregular. In certain embodiments, the nanofiber sheet is cut to provide implants that are rod-shaped.

In certain embodiments, a method of making an ocular implant comprising a plurality of nanoparticles comprises:
dissolving a drug (e.g., water-soluble drug, such as pregabalin) and one or more bioresorbable polymers in a solvent to provide a solution,
electrospraying the solution to provide a plurality of nanoparticles on a collector surface,
collecting the plurality of nanoparticles from the collector surface, and
compressing at least a portion of the plurality of nanoparticles to form the implant; wherein
the nanoparticles provide for sustained release of the drug from the implant; and
the implant is suitable for implantation into the eye of a mammal.

The bioresorbable polymers described hereinabove can be used in the method provided. In certain embodiments, the one or more bioresorbable polymers comprise poly(L-lactic acid) (PLA), polycaprolactone (PCL), polyglycolic acid, or a copolymer thereof. In certain embodiments, the one or more bioresorbable polymers comprise poly(L-lactic acid) (PLA). In certain embodiments, the one or more bioresorbable polymers comprise polycaprolactone (PCL). In certain embodiments, the one or more bioresorbable polymers comprise poly(L-lactic acid) (PLA). In certain embodiments, the one or more bioresorbable polymers comprise a copolymer of poly(L-lactic acid) (PLA), polycaprolactone (PCL), and polyglycolic acid.

The drug (e.g., water-soluble drug) and one or more bioresorbable polymers are dissolved in any solvent in which both components are soluble. Exemplary solvents include organic solvents, organic acid solvents, inorganic acid solvents, and water.

In certain embodiments, the solvent comprises an organic solvent. In certain embodiments, the organic solvent comprises chloroform, acetone, dichloromethane, dimethylformamide, ethyl acetate, tetrahydrofuran, dimethyl sulfoxide, alcohol (e.g., methanol, ethanol, propanol, isopropanol, trifluoroethanol, hexafluoroisopropanol), or a combination thereof.

In certain embodiments, the solvent comprises an organic acid solvent. In certain embodiments, the organic acid solvent comprises acetic acid, trifluoroacetic acid, propionic acid, or a combination thereof. In certain embodiments, the organic acid solvent comprises acetic acid. In certain embodiments, the organic acid solvent comprises glacial acetic acid. In certain embodiments, the organic acid solvent consists of glacial acetic acid.

In certain embodiments, the solvent comprises an organic solvent. In certain embodiments, the organic solvent comprises chloroform, acetone, dichloromethane, dimethylformamide, ethyl acetate, methanol, ethanol, or a combination thereof. In certain embodiments, the organic solvent comprises methanol.

In certain embodiments, the solvent comprises less than 5 wt % water, such as, for example, from 0 wt % to 5 wt %, from 0 wt % to 3 wt %, from 0 wt % to 1 wt %, from 0.1 wt % to 5 wt %, from 0.1 wt % to 3 wt %, from 0.1 wt % to 1 wt %, from 0.5 wt % to 5 wt %, from 0.5 wt % to 3 wt %, or from 0.5 wt % to 1 wt %.

In certain embodiments, the drug (e.g., water-soluble drug or water-insoluble drug) and bioresorbable polymer are combined in a weight ratio of 1:1 to 1:20 in the solution, such as, for example, from 1:1 to 1:15, from 1:1 to 1:10, from 1:1 to 1:5, from 1:2 to 1:6, from 1:2 to 1:5, from 1:2 to 1:4, from 1:2 to 1:3, from 1:3 to 1:6, from 1:3 to 1:5, from 1:3 to 1:4, from 1:4 to 1:6, or from 1:4 to 1:5. In certain embodiments, the weight ratio of drug to one or more bioresorbable polymer is 1:4.

In certain embodiments, dissolving the drug (e.g., water-soluble drug, such as pregabalin) and one or more bioresorbable polymers in a solvent to provide a solution is conducted by heating a mixture of the drug and bioresorbable polymer to promote dissolution. In certain embodiments, the mixture is heated to 30° C. to 100° C., such as, for example, from 30° C. to 75° C., from 30° C. to 50° C., from 50° C. to 100° C., from 50° C. to 75° C., or from 75° C. to 100° C.

In certain embodiments, the drug and bioresorbable polymer is mixed, e.g., shaken or stirred, to promote dissolution.

Once the solution of the drug and one or more bioresorbable polymers is prepared, the solution is electrosprayed using an electrosprayer, e.g., a Spinbox from NanoScience Instruments (Phoenix, AZ), to provide a plurality of nanoparticles deposited on a collector surface (e.g., the collector surface of the electrosprayer). A person of skill in the art will understand that the parameters of the electrosprayer will vary depending on the components being electrosprayed and their relative amounts.

In some embodiments, the solution is electrosprayed at a flow rate of 1 μl/min to 20 μl/min, such as, for example, from 1 μl/min to 15 μl/min, from 1 μl/min to 10 μl/min, from 5 μl/min to 20 μl/min, from 5 μl/min to 15 μl/min, from 5 μl/min to 10 μl/min, from 10 μl/min to 20 μl/min, from 10 μl/min to 15 μl/min, or from 15 μl/min to 20 μl/min. In certain embodiments, the solution is electrosprayed at a flow rate of 8 μl/min.

In certain embodiments, the distance between the tip of the spray nozzle of the electrosprayer and the collector surface is from 5 cm to 20 cm, such as, for example, from 5 cm to 15 cm, from 5 cm to 10 cm, from 10 cm to 20 cm, from 10 cm to 15 cm, from 15 cm to 20 cm, or from 13 cm to 19 cm.

In certain embodiments, the applied potential difference between the spray nozzle and the collector surface is from 10 kV to 22 kV, such as, for example, from 10 kV to 20 kV, from 10 kV to 15 kV, from 15 kV to 22 kV, or from 15 kV to 20 kV. In certain embodiments, the applied potential difference is 17 kV.

Electrospraying provides a dry powder comprising a plurality of nanoparticles. In certain embodiments, a portion or all of the plurality of nanoparticles deposited on the electrosprayer collector surface are collected and compressed to form an implant. The compression can be performed with any suitable device, e.g., a tablet press or die. As a person of skill in the art will understand, the particular device and settings of the device will vary depending on the desired implant shape. In certain embodiments, compression provides one of the following implant shapes: spherical, oblate, ellipsoidal, rod, disc, tube, hemispherical, or irregular. In certain embodiments, the compression provides a rod-shaped implant.

In certain embodiments, the method further comprises filing the implant to remove rough edges. The filing can be done with any suitable device, e.g., a file or sandpaper.

In certain embodiments, the methods for preparing nanoparticle implants or nanofiber implants described herein further comprise applying a polymer coating to the implant. The polymer coating can be applied by any suitable device, e.g., a coater drier or mini-coater drier. In certain embodiments, the polymer coating comprises polyethylene glycol, a zwitterionic polymer, a hydrophobic bioresorbable polymer, a bioadhesive polymer, or a combination thereof.

In certain embodiments, the polymer coating comprises a zwitterionic polymer. In certain embodiments, the zwitterionic polymer is selected from poly(sulfobetaine methacrylate), poly(carboxybetaine methacrylate), and a combination thereof.

In certain embodiments, the polymer coating comprises a bioresorbable polymer. In certain embodiments, the polymer coating is the same as the bioresorbable polymer described hereinabove. In certain embodiments, the bioresorbable polymer coating is different than the bioresorbable polymer described hereinabove.

In certain embodiments, the polymer coating comprises a hydrophobic bioresorbable polymer. In certain embodiments, the hydrophobic bioresorbable polymer comprises polycaprolactone (PCL).

In certain embodiments, the polymer coating comprises a bioadhesive polymer. In certain embodiments, the bioadhesive polymer is selected from a polyacrylic acid derivative, cellulose derivative, natural polymer, polyvinyl pyrrolidone (PVP), dextran polymer, polyethylene oxide polymer, thermoreversible polymer, ionic responsive polymer, copolymer of polymethyl vinyl ether and maleic anhydride, and a combination thereof.

In certain embodiments, the polymer coating comprises a bioadhesive, natural polymer. In certain embodiments, the bioadhesive, natural polymer is selected from gum arabic, tragacanth gum, agar polymer, xanthan gum, copolymer of alginic acid and sodium alginate, chitosan polymer, pectin, carrageenan, pullulan polymer, modified starch, and a combination thereof.

In certain embodiments, the polymer coating comprises a biocompatible polymer, e.g., a Eudragrit. Eudragits are, generally, acrylate-methacrylate copolymers, optionally including quaternary ammonium groups, that are commercially available from Rohm Pharma under the tradename Eudragit®. Exemplary Eudragits include those suitable for sustained release, e.g., Eudragit® RL PO, Eudragit® RL 100, Eudragit® RL 30 D, Eudragit® RL 12.5, Eudragit® RS PO, Eudragit® RS 100, Eudragit® RS 30S, and Eudragit® 12.5.

In certain embodiments, the methods for preparing nanoparticle implants or nanofiber implants described herein further comprise sterilizing the implant. Any suitable method of sterilization can be used, e.g., ethylene oxide, gamma radiation, or e-beam radiation.

### 4. METHODS OF TREATMENT

In one aspect of the present disclosure, a method of treating an ocular condition in a subject afflicted with an ocular condition is provided, the method comprising placing an implant described herein in the eye of the subject.

The implant can be placed in any functional (e.g., for vision) or structural tissues found in the eye, or tissues or cellular layers that partly or completely line the interior or exterior of the eye. Specific examples of areas of the eye include the anterior chamber, the posterior chamber, the vitreous cavity, the choroid, the suprachoroidal space, the conjunctiva, the subconjunctival space, the episcleral space, the intracorneal space, the epicorneal space, the sclera, the pars plana, surgically induced avascular regions, the macula, and the retina.

In certain embodiments, the implant is placed in one of the following locations: the subconjunctival space, the anterior chamber, the posterior chamber, the intravitreal chamber, the subtenon space, the inferior palpebral sulcus, the superior palpebral sulcus, the suprachoroidal space, or the intracameral space. In certain embodiments, the implant is placed in the subconjunctival space of the eye. In certain embodiments, the implant is placed in the anterior chamber of the eye.

In other embodiments, the implant is placed elsewhere on the subject's body other than the eye, e.g., subcutaneously or intradermally to provide long term control of systemic diseases.

For subconjunctival placement of the implant, in some instances, a pocket is made by creating an incision at the inferior fornix of the eye. The implant is then placed in the pocket. The implant is placed by injection with, e.g., a syringe with a needle. The gauge (G) of the needle should be suitable for injection to the desired location in the eye. In certain embodiments, a syringe fitted with a 19G to 25G needle is used. In certain embodiments, a syringe fitted with a 21G needle is used for injection of the implant into the subconjunctival space. In certain embodiments, a syringe fitted with a 30G needle is used for injection into the intravitreal space. Once the implant is placed in the subconjunctival space, the incision is closed, such as by suturing.

In certain embodiments, for subconjunctival placement, the implant is injected with a syringe. In such embodiments, no incision or suture step is required.

In certain embodiments, the subject is administered topical antibiotics following placement of the implant to prevent infection.

In certain embodiments, an implant is placed in one eye of the subject. In certain embodiments, one implant is placed in each eye of the subject.

In certain embodiments, the implant provides sustained release as described hereinabove for treatment of an ocular condition. In certain embodiments, the ocular condition is selected from elevated IOP, glaucoma, intraocular inflammation, keratitis, dry eye, macular edema, diabetic macular edema (DME), infection, macular degeneration, age-related macular degeneration (AMD), blurred vision, herpetic conjunctivitis, blepharitis, retinal or choroidal neovascularization, uveitis, diabetic retinopathy, ischemic retinopathy, optic neuropathy, ocular cancer, chronic ocular allergic conditions, and cataract.

In certain embodiments, the ocular condition is elevated IOP. In certain embodiments, the ocular condition is glaucoma-associated elevated IOP. In certain embodiments, the ocular condition is glaucoma.

In certain embodiments, the present methods reduce the TOP of the subject by at least 10% from baseline measurement, such as, for example, at least 20% lower than baseline, at least 30% lower than baseline, at least 40% lower than baseline, at least 50% lower than baseline, at least 60% lower than baseline, at least 70% lower than baseline, at least 80% lower than baseline, at least 90% lower than baseline, or at least 100% lower than baseline.

In certain embodiments, the methods reduce IOP from baseline by at least 2 mm Hg, at least 3 mm Hg, at least 4 mm Hg, at least 5 mm Hg, at least 6 mm Hg, at least 7 mm Hg, at least 8 mm Hg, at least 9 mm Hg, at least 10 mm Hg, at least 11 mm Hg, at least 12 mm Hg, at least 13 mm Hg, at least 14 mm Hg, or at least 15 mm Hg.

In certain embodiments, the present methods reduce IOP from greater than 21 mm Hg at baseline to 21 mm Hg or less during treatment, such as, for example, from 21 mm Hg to 10 mm Hg, from 20 mm Hg to 10 mm Hg, from 19 mm Hg to 10 mm Hg, from 18 mm Hg to 10 mm Hg, from 17 mm Hg to 10 mm Hg, from 16 mm Hg to 10 mm Hg, from 15 mm Hg to 10 mm Hg, from 14 mm Hg to 10 mm Hg, or from 13 mm Hg to 10 mm Hg.

In certain embodiments, the reduction in IOP is maintained for a continuous period of time of at least 6 months, such as, for example, at least 7 months, at least 8 months, at least 9 months, at least 10 months, at least 11 months, at least 12 months, at least 13 months, at least 14 months, at least 15 months, at least 18 months, or at least 24 months from the time of implantation. In certain embodiments, the reduction in IOP is maintained for at least 12 months from the time of implantation.

In certain embodiments, the variability of IOP over the course of treatment is less than 5 mm Hg, such as, for example, less than 4 mm Hg, less than 3 mm Hg, less than 2 mm Hg, or less than 1 mm Hg.

In certain embodiments, the methods further comprise placing one or more subsequent implants after the initial implant(s) are spent. In certain embodiments, one implant is placed in the eye of the subject every 6 months, 7 months, 8 months, 9 months, 10 months, 11 months, 12 months, 13 months, 14 months, 15 months, 18 months, or 24 months. In certain embodiments, one implant is placed in each eye of the subject every 6 months, 7 months, 8 months, 9 months, 10 months, 11 months, 12 months, 13 months, 14 months, 15 months, 18 months, or 24 months.

### 5. EXAMPLES

### Example 1: Preparation of Implants

For both nanoparticle and nanofiber implants, pregabalin and polymer were dissolved in solvents or solvents systems in the amounts provided in Table 1. The solution was packed in a 20 ml syringe then connected to the Spinbox infusion pump. The solution was electrosprayed (for nanoparticles) or electrospun (for nanofibers) using Spinbox electro-sprayer/spinner at flow rate 8 μl/min. The distance between the spray nozzle and the collector was kept between 13 cm and 19 cm. The applied potential difference between the spray nozzle and the collector was kept at 17 kV. The product was collected from the collector surface after all the solution was sprayed (~40 hour). For nanoparticles, the product was collected by a spatula in powder form and kept in an airtight glass bottle until further analysis. Nanofibers were collected as one piece (in the form of sheet) and kept in an air-tight glass container until further analysis. It is noted that Eudragit is not a bioresorbable polymer but was used in Table 1 as a proof-of-concept biocompatible polymer to demonstrate formation of nanoparticles.

TABLE 1
Composition of Electrospraying and Electrospinning solutions

| | Formulation number | | | | | | | | | | | | | | | | | | | | | |
|---|---|---|---|---|---|---|---|---|---|---|---|---|---|---|---|---|---|---|---|---|---|---|
| Ingredients (wt %) | F₁ | F₂ | F₃ | F₄ | F₅ | F₆ | F₇ | F₈ | F₉ | F₁₀ | F₁₁ | F₁₂ | F₁₃ | F₁₄ | F₁₅ | F₁₆ | F₁₇ | F₁₈ | F₁₉ | F₂₀ | F₂₁ | F₂₂ |
| Pregabalin | 2.5 | 2.5 | 5 | 3 | 3 | 0.9 | 5 | 5 | 5 | 5 | 5 | 5 | 1 | 5 | 5 | 5 | 5 | 5 | 5 | 5 | 5 | 5 |
| Low M. wt. HPMC | 4 | — | — | — | — | — | — | — | — | — | — | — | — | — | — | — | — | — | — | — | — | — |
| High M. wt. HPMC | — | 4 | — | — | — | — | — | — | — | — | — | — | — | — | — | — | — | — | — | — | — | — |
| Eudragit RL100 | — | — | 10 | 10 | 10 | 10 | 20 | 20 | 20 | 20 | 20 | 20 | 1 | — | — | — | — | — | — | — | — | — |
| Soybean Lecithin | — | — | — | — | — | — | — | — | — | — | — | — | 8.5 | — | — | — | — | — | — | — | — | — |
| Poloxamer 407 | — | — | — | — | — | — | — | — | — | — | — | — | 8.5 | — | — | — | — | — | — | — | — | — |
| Polylactide (PLA) | — | — | — | — | — | — | — | — | — | — | — | — | — | — | — | — | — | — | 20 | — | — | — |
| Polylactide-co-glycolide | — | — | — | — | — | — | — | — | — | — | — | — | — | — | — | — | — | — | — | 20 | — | — |
| (PLGA) | | | | | | | | | | | | | | | | | | | | | | |
| Poly-ε-caprolactone | — | — | — | — | — | — | — | — | — | — | — | — | — | — | — | — | — | — | — | — | 20 | — |
| (PCL) | | | | | | | | | | | | | | | | | | | | | | |
| Poly(lactide-co- | — | — | — | — | — | — | — | — | — | — | — | — | — | — | — | — | — | — | — | — | — | 20 |
| caprolactone-co- | | | | | | | | | | | | | | | | | | | | | | |
| glycolide | | | | | | | | | | | | | | | | | | | | | | |
| Poly(lactide-co- | — | — | — | — | — | — | — | — | — | — | — | — | — | 20 | 20 | 20 | 20 | 20 | — | — | — | — |
| caprolactone | | | | | | | | | | | | | | | | | | | | | | |
| Ethanol/Water (3:1) | 93.5 | 93.5 | | | | | | | | | | | | | | | | | | | | |
| Methanol | | | 85 | 87 | | | 75 | | | | | | | | 75 | | | | | | | |
| Ethanol | | | | | 87 | | | | | | | | | | | | | | | | | |
| Methanol/Water (3:1) | | | | | | 89.1 | | | | | | | | | | | | | | | | |
| Chloroform | | | | | | | | 75 | | | | | | 75 | | | | | | | | |
| Acetone | | | | | | | | | 75 | | | | | | | | | | | | | |
| Dichloromethane | | | | | | | | | | 75 | | | | | | | | | | | | |
| Dimethylformamide | | | | | | | | | | | 75 | | | | | | | | | | | |
| Ethyl Acetate | | | | | | | | | | | | 75 | | | | | | | | | | |
| Acetone/Ethanol (1:1) | | | | | | | | | | | | | 81 | | | | | | | | | |
| DCM/Methanol (9:1) | | | | | | | | | | | | | | | | 75 | | | | | | |
| Tween 80/ | | | | | | | | | | | | | | | | | 73 | | | | | |
| Methanol/Acetone | | | | | | | | | | | | | | | | | | | | | | |
| (2:10:88) | | | | | | | | | | | | | | | | | | | | | | |
| Glacial acetic acid | | | | | | | | | | | | | | | | | | 75 | 75 | 75 | 75 | 75 |
| Form | NP | NF | NP | NP | NP | NP | NP | — | — | — | — | — | — | — | — | — | — | NF | — | — | — | — |

Some pregabalin/polymer/solvent compositions of Table 1 (marked with “—” for “Form”) proved unsuitable for electrospinning or electrospraying or did not result in usable nanostructures upon electrospinning or electrospraying.

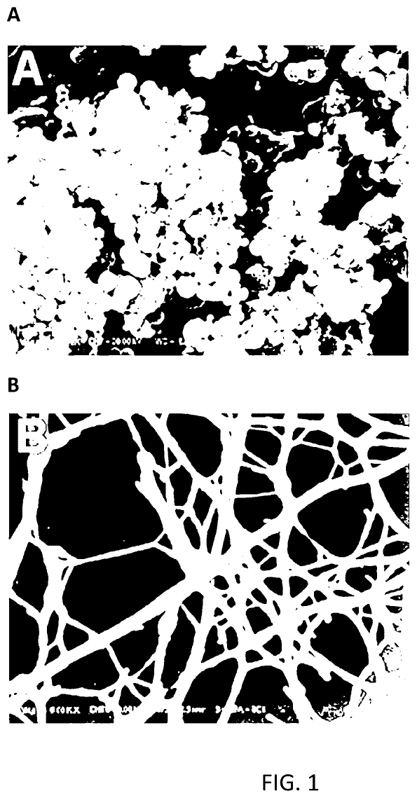

The external structure of the obtained nanofibers and nanoparticles was examined using scanning electron microscopy (SEM) with a Carl Zeiss EVO HD LS15 (LaB6) with VPSE, STEM Detectors and Oxford EDS detector (FIG. 1).

For nanofibers, the obtained sheet was cut into 5 mg weight rod-shape implants with the dimensions of (4.0×2.0×0.5 mm, L×W×H) using a cutting punch. For nanoparticles, the powder was compressed into rod-shape tablets with the dimensions of (6 length and 0.5 mm diameter) using Vice hand-held tablet press.

### Example 2: Physical Properties of the Implant

Characterization of physical properties of pregabalin nanofiber and nanoparticle implants was conducted by measuring pH using a pH meter and assessing particle size, polydispersity index (PDI) and zeta potential measurements using Zetasizer. Pregabalin nanofiber and nanoparticle implants were evaluated in vitro by determining the uniformity of drug content by HPLC.

Table 2 details properties of the implants prepared according to Example 1.

TABLE 2
Properties of implant compositions

| | Implant Formulation | | |
|---|---|---|---|
| | PRG- | PRG- | |
| | Eudragit (1:4) | Eudragit (1:11.5) | PRG-PLA-PCL |
| Parameter | nanoparticles | nanoparticles | nanofiber rod |
| pH | 7.2 ± 0.02 | 7.10 ± 0.11 | 5.2 ± 0.15 |
| Particle Size (nm) | 957.67 ± 69.44 | 906.90 ± 63.62 | 312.6 ± 89.3* |
| PDI | 0.69 ± 0.01 | 0.64 ± 0.01 | — |
| Zeta Potential (mV) | 31.27 ± 0.78 | 31.62 ± 1.11 | 32.90 ± 1.10 |
| Uniformity of drug | 96.95 ± 4.23 | 103.42 ± 1.82 | 94.88 ± 1.6 |
| content | | | |

With respect to Table 2, “Particle size” refers to particle size for the nanoparticle implant formulations. “Particle Size” refers to the diameter of the nanofiber for the nanofiber formulation.

The pH of the nanoparticles and nanofibers were measured using a pH meter by placing the nanoparticles or nanofibers in water and measuring the pH of the water (Corning pH meter 440; Corning Inc., Corning, New York). Half a gram of nanoparticles or nanofibers were dispersed in 10 mL of Milli-Q water at room temperature for 2h, and then the pH was measured. The experiment was repeated three times, and the results were presented as mean±SEM.

Particle size, polydispersity index (PDI) and zeta potential of the nanoparticles were determined after suitable dilution using Zetasizer (Nanoseries, nano-ZS, Malvern Instruments Limited, UK). All measurements were performed at 25° C. The results of three independent test runs were presented as mean±SEM.

Uniformity of drug content was measured by randomly selecting ten implants. Each implant was individually dissolved in 10 ml of a common solvent for both the drug and the polymer from which the implant was made. After complete dissolution, the solution was filtered through 0.22 mm membrane filter and assayed for its drug content using UV-HPLC and the amount of drug content calculated from the standard curve. The individual implant drug content was calculated as a percentage from the labeled amount.

Characterization of physical properties of pregabalin nanofiber and nanoparticle implants illustrated that both implants have pH values in the range that can be tolerated by the eye. Both implants had a relatively large particle size that is expected to decrease the surface area and support the sustained slow PRG release after subconjunctival application. Both implants showed a high zeta potential value which is indicative of excellent shelf stability. In addition, pregabalin nanofiber and nanoparticle implants had an adequate drug content in the acceptable pharmacopeial range (90%-110%) which is indicative of the minimal loss of drug during the implant preparation.

### Example 3: In Vitro Release

The drug release behavior from the rods was evaluated as described in the following references:
Ibrahim, M., Abd-Elgawad, A., Soliman, O. & Jablonski, M. “Nanoparticle-based topical ophthalmic formulations for sustained celecoxib release.” J Pharm Sci 102, 1036-1053 (2013).
Ibrahim, M., Abd-Elgawad, A., Soliman, O. & Jablonski, M. “Stability and Ocular Pharmacokinetics of Celecoxib-Loaded Nanoparticles Topical Ophthalmic Formulations.” J Pharm Sci 105, 3691-3701 (2016).
Ibrahim, M., Abd-Elgawad, A., Soliman, O. & Jablonski, M. “Novel topical ophthalmic formulations for management of glaucoma.” Pharm Res 30, 2818-2831 (2013).
Ibrahim, M., Abd-Elgawad, A., Soliman, O. & Jablonski, M. “Natural bioadhesive biodegradable nanoparticles-based topical ophthalmic formulations for sustained celecoxib release: In vitro Study.” Pharm Technol & Drug Res 2, 7 (2013).

Briefly, the sustained release behaviors of pregabalin from different implants were studied in PBS (pH 7.4) using 1500 μl fast micro-equilibrium dialyzer (Harvard Apparatus Co., Holliston, MA) with semipermeable regenerated cellulose membranes (Molecular weight cut off 1,000 Da) separating two chambers. The implant was placed in the first chamber, and the second chamber contained PBS (pH 7.4). The dialyzers were kept in a thermostatically controlled shaker at 37° C. and 50 rpm. Samples were withdrawn at predetermined time intervals for 24 h and analyzed for pregabalin content by HPLC.

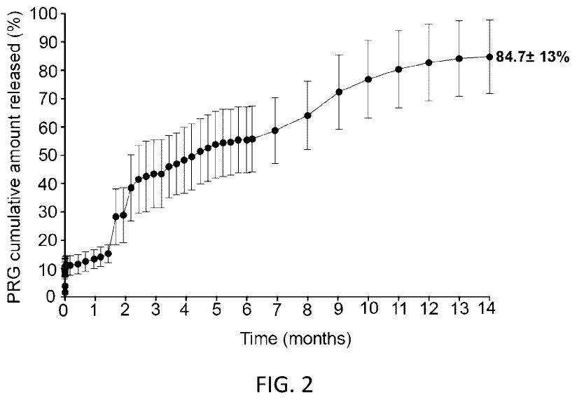

FIG. 2 shows the pregabalin release profile for the PRG-PLA-PCL (pregabalin: DL-Lactide-co-caprolactone) nanofiber implant over fourteen months. The implant had an average release of 1.38±0.25 μg/day after an initial burst of 8.0±1.6% within the first hour, which equates to 84.7±13.0% of its cargo over a period of over fourteen months (mean±SEM; n=3). Extrapolation of these data suggest multiple months of Higuchi Fickian release kinetics, assuming that drug release proceeds to 100% with no change in rate as the implant degrades. These data demonstrate that the PRG-PLA-PCL nanofiber implants can sustain and control PRG in vitro release over more than a year. In addition, these promising in vitro release data of the PRG-PLA-PCL nanofiber implants supports its once yearly application. Without wishing to be bound by theory, it is postulated that one or both of the nanofiber web-like architecture and the avoidance of compression of the nanostructures in preparing the implant may lead to the extended release times observed for the nanofiber implants disclosed herein.

### Example 4: Sclera/Choroid Permeability

The permeability, steady-state flux, and permeation rate of pregabalin across the sclera/choroid can be measured using published methods, e.g., as described in PCT/US2018/066235. Modified Franz diffusion cell consists of two vertically connected chambers (donor and receptor chambers) attached to each other through a spherical junction that is suitable for spherical tissues (i.e., sclera). The volume of the donor chamber is 0.5 ml and that of the receptor chamber is 5 ml. The receptor chamber is stirred at 50 rpm by the aid of a magnetic stir bar to allow continuous mixing of the chamber contents. The orifice at the junction between the two chambers has a 9 mm diameter at which the sclera/conjunctiva tissue is fixed, the implant is sandwiched between sclera and conjunctiva and the drug allowed to diffuse through the sclera to the receptor chamber. One implant is placed in the donor chamber. The receptor chamber is filled with 5 ml of BSS-Plus (balanced salt solution-plus) and the whole cell surrounded by a warm water jacket to maintain the temperature at 37° C. A sample of 0.5 ml is withdrawn from the receptor chamber every 1 h for a total period of 5 h and replaced by a fresh BSS-Plus that maintained at 37° C. The collected samples are assayed for their drug contents using a standard HPLC protocol. The experiment can be repeated six times and the results calculated as mean±SEM. The data are plotted as cumulative amount permeated (μg) versus time. From the slope of that plot, permeation rate, steady-state flux and permeability coefficient are calculated.

Ex vivo trans-scleral permeability of PRG-PLA-PCL nanofiber implants (PRG-NF) and PRG-Eudragit nanoparticle implants (PRG-NP) implants was measured as outlined below.

General procedure:

Freshly separated conjunctiva and sclera from New Zealand white rabbit eyes were used during this study. PRG implants were sandwiched between the conjunctiva and sclera that mounted on modified rounded-junction Franz diffusion cells. The temperature of the cells was maintained at 37±0.5° C. with the help of a circulating water bath. The receptor chamber was continuously stirred and filled with 5 mL of balanced salt solution enriched with bicarbonate, dextrose, and glutathione, BSS-PLUS. At predetermined time intervals (1, 2, 3, 4, and 5 h), 500 μL were withdrawn from the receptor chamber and replaced with an equal volume of fresh warmed BSS-PLUS. The concentration of PRG permeated in withdrawn samples was then immediately determined using an UV-HPLC. Both implants deliver PRG through the sclera of New Zealand white rabbits at a controlled rate.

The ex-vivo trans-scleral permeability parameters of PRG-loaded nanofiber and nanoparticle implants are presented in Table 3.

TABLE 3
Ex-vivo trans-scleral permeability parameters of
PRG-loaded nanofiber and nanoparticle implants.

| | Rate of | | Permeability |
|---|---|---|---|
| | permeation | Flux | coefficient |
| Uncoated | (dM/dt) | (μg/ | (P) × 10⁻⁴ |
| Formulation | (μg/min) | cm² · min) | (cm/min) |
| PRG-NF implants | 0.251 ± 0.06 | 0.007 ± 0.002 | 0.053 ± 0.013 |
| PRG-NP implants | 0.759 ± 0.302 | 0.020 ± 0.008 | 0.022 ± 0.009 |

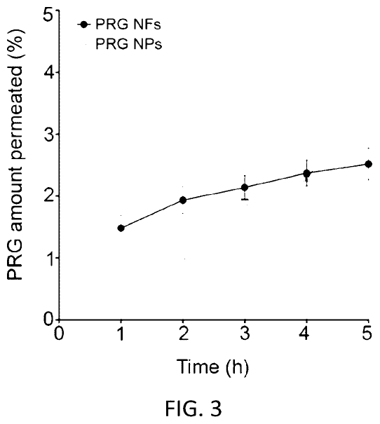

The results are shown in FIG. 3. PRG-NF and PRG-NP implants control the high permeability of PRG (BCS class I drug) and suggest the ability of the implants to delivery drug through 12 months at a constant rate.

### Example 5: In Vivo Efficacy, Safety, and Biocompatibility

IOP-lowering efficacy, safety, and biocompatibility of the PRG implant are measured when placed in the subconjunctival space of anesthetized Dutch belted (DB) rabbits, a well characterized pre-clinical model with spontaneously elevated IOP (Hays, C., Okafor, K., High, R., Singh, D. & Toris, C. Consequences of Puberty on Efficacy of IntraocularPressure-Lowering Drugs in Male Dutch-Belted Rabbits. J Ocul Pharmacol Ther 34, 76-84 (2018)). This study is a non-GLP, prospective, randomized, parallel-arm, masked design with two PRG doses (0.5 mg/implant and 2 mg/implant to compare responses to dose levels) and with 4 controls: 1) sham surgery; 2) placebo implant; 3) a PRG microemulsion (0.6%) (e.g., PCT/US2018/066235); and 4) an active comparator control of Latanoprost. Male and female rabbits (2 each) are evaluated under each experimental condition. Efficacy, safety, and biocompatibility are determined as outlined below. Primary outcome measures include IOP changes, anterior/posterior segment parameters, and histocompatibility. The number and severity of adverse events are recorded.

Implant insertion: Using a lid speculum, conjunctival forceps and a sterile field, 1 implant per eye (4 mm×2 mm×0.5 mm inserted longitudinally; both eyes of each rabbit receive the same implant) is placed in a surgical pocket created by a 2.5 mm incision at inferior fornix of the eye. After placement of the implant, the incision is closed with 10-0 nylon sutures and eyes given topical antibiotics.

Efficacy: To determine efficacy, IOP is measured using a Tono-pen Avia tonometer and published methods (Ibrahim, M. et al. Once Daily Pregabalin Eye Drops for Management of Glaucoma. ACS Nano 13, 13728-13744 (2019)). Readings are taken before enrollment into the study and daily for 2 weeks after insertion of the implant. Thereafter, IOP is assessed weekly. Eyes are grossly examined for inflammation, swelling, irritation, etc. with each IOP reading. If there are any adverse reactions, rabbits are evaluated using the safety protocol below. IOP for 1 month longer than PRG is released from the implant is measured so that any physiological effects that continue beyond drug release are discerned.

Safety and biocompatibility: Safety and biocompatibility of the PRG implant along with the four above-listed controls are determined using: the SPOTS system (Eaton, J., Miller, P., Bentley, E., Thomasy, S. & CJ, M. The SPOTS System: An Ocular Scoring System Optimized for Use in Modern Preclinical Drug Development and Toxicology. J. Ocular Pharmacol Ther 33, 718-734 (2017)); inlife slit lamp biomicroscopic examinations; and histopathology. Clinical examinations are performed on each rabbit before study enrollment in addition to the following times after implant insertion: 1 day; 1 week; and monthly until study conclusion.
Anterior segment examinations using a slit lamp biomicroscope: pupillary light reflex; conjunctival hyperemia, swelling and hyperemia; corneal opacity and vascularization; anterior chamber cells and flare, iris involvement, anterior vitreous cell infiltration; corneal fluorescein staining severity and area; and lens opacity. Although not a component of the SPOTS system, each eye is examined to determine if the implant has moved, and for any indication of inflammation, rejection, etc., both at and away from the site of implant insertion.
Posterior segment examinations using a slit lamp biomicroscope and a 78-diopter lens: vitreous haze; degraded fundus view; and retinal perivascular sheathing.
Histopathology: After each cohort of rabbits is sacrificed the following ocular tissues are processed for histopathology: full thickness globe at the site of the implant including a 2 mm rim; iridocorneal angle including ciliary body and outflow structures; and central & peripheral retina. Sections obtained from the area surrounding the implant are stained with Masson Trichrome and CD45 to determine foreign body response, whether a collagen capsule is formed, and whether T cells are infiltrated, respectively. The girth of the implant is measured to estimate the amount of implant remaining. To determine if the endothelium is negatively impacted, corneas are hemisected and half are stained with Alizarin red S to calculate the density of corneal endothelium.

Biodistribution of PRG: These studies determine the distribution of drug in the eye at one month after implant insertion and at study conclusion. The following tissues are analyzed: conjunctiva, sclera and choroid (at the site of insertion and at 180°); aqueous humor; cornea; lens; ciliary body; trabecular meshwork; vitreous; and the retina less portion taken for histopathology. The amount of PRG in tissues is compared between treatments: PRG-loaded implant; placebo implant; sham surgery; and PRG ME.

Rigor, statistical analyses, and sex as a variable: Assuming a power of 80%, and a meaningful marginal difference in IOP of 25%, it is estimated that this study requires 8 eyes for each experimental condition tested. Equal numbers of male and female rabbits are included and responses from each sex will initially be evaluated separately and combined if there are no significant differences. To ensure rigor in clinical evaluations, between 3-5 measurements are collected from each test per eye per rabbit, averaged and considered one biological sample. To reduce bias, the experimenter is blinded as to which contents of the formulation used to dose each eye. Both quantitative and semi-quantitative values are expressed as mean±SEM. Statistical differences between groups are analyzed by one-way analysis of variance (ANOVA) followed by Tukey's post hoc test (in the event of a significant F-test) using GraphPad Prism software. Significance is set at p<0.05.

### Example 6: Swelling Behavior of Pregabalin Nanofiber and Pregabalin Nanoparticle Implants

The swelling behavior of PRG-PLA-PCL nanofiber (PRG-NF) and PRG-Eudragit nanoparticle (PRG-NP) implants was investigated as outlined below.

### General Procedure:

The swelling index of the prepared pregabalin nanofiber (PRG-NF) and pregabalin nanoparticle (PRG-NP) depot implants was determined using agar gel plate method. A plate containing 15 mL of 2% w/v agar solution was prepared and allowed to solidify at room temperature. PRG implants were weighed initially using analytical balance and kept on the surface of the agar gel plate at 37±1° C. afterwards. PRG implants were reweighed using analytical balance after several time intervals (1 h, 2 h, 6 h, 24 h, 48 h, and 72 h). The agar gel plate was kept covered with a glass cover during the experiment.

The swelling index was calculated according to the following equation:

$$swelling percentage = [ ( weight of swollen implant - weight of initial implant ) / weight of initial implant ] \times 100$$

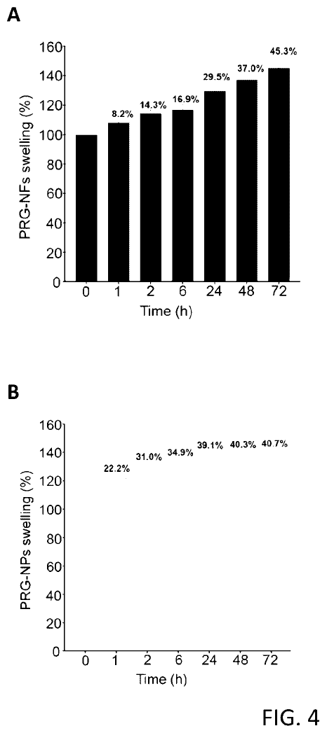

FIG. 4, panels A and B, present the results. The PRG-NF and PRG-NP implants demonstrated high swelling at the first few hours. The swelling equilibrates after the first day (mean±SEM; n=6).

Such swelling behavior is expected to support drug release at a constant rate after administration.

### Example 7: Moisture Uptake of Pregabalin Nanofiber and Pregabalin Nanoparticle Implants

Moisture uptake behavior of PRG-PLA-PCL nanofiber implants (PRG-NF) and PRG-Eudragit nanoparticle implants (PRG-NP) was investigated to assess the implant shelf stability at room temperature.

General procedure:

PRG-NF and PRG-NP implants were stored in a desiccator with 79.5% relative humidity (RH). RH was generated by a saturated solution of ammonium chloride in water to determine the ability of PRG-NF and PRG-NF implants to absorb moisture to test stability during shelf-life storage at room temperature (24±1° C.). PRG-NF and PRG-NP implants were weighed using analytical balance initially and after three days of storage. The moisture uptake was determined using the following equation:

$$moisture uptake ( \% ) = [ ( final weight of implant - initial weight of implant ) / initial weight of implant ] \times 100$$

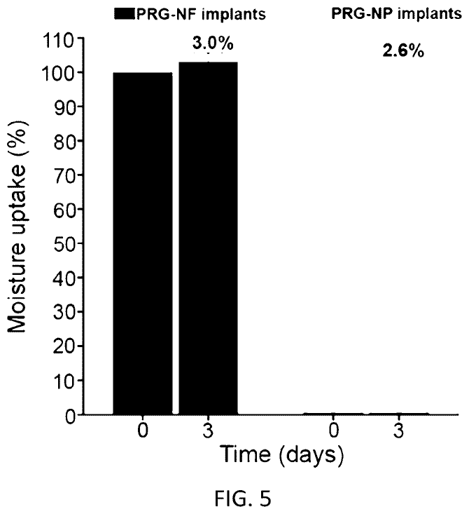

FIG. 5 presents the results of the moisture uptake test over three days. Both PRG-NF and PRG-NP implants were stable and had a minimal moisture uptake (mean±SEM; n=6). These results are indicative of adequate shelf-life stability of both PRG-NF and PRG-NP implants.

### 6. EQUIVALENTS AND INCORPORATION BY REFERENCE

While the provided disclosure has been particularly shown and described with reference to a preferred embodiment and various alternate embodiments, it will be understood by persons skilled in the relevant art that various changes in form and details can be made therein without departing from the spirit and scope of the provided disclosure.

All references, issued patents, and patent applications cited within the body of the instant specification, are hereby incorporated by reference in their entirety, for all purposes. In particular, PCT Patent Application No. PCT/US2018/066235 (filed Dec. 18, 2018), PCT Patent Application No. PCT/US2021/047109 (filed Aug. 23, 2021), and U.S. Provisional Patent Application No. 63/579,826 (filed Aug. 31, 2023) are hereby incorporated by reference in their entirety.

1-118. (canceled)

119. A biodegradable implant configured for intravitreal administration of an intraocular pressure (IOP)-lowering drug to a subject, the implant comprising:
(i) 0.1-50 wt % of the IOP-lowering drug; and
(ii) 50-99.9 wt % of one or more bioresorbable polymers;
wherein the one or more bioresorbable polymers form a polymer matrix containing the IOP-lowering drug; and
wherein the matrix provides for sustained release of the IOP-lowering drug in an intravitreal space of the subject.

120. The implant of claim 119, wherein the IOP-lowering drug is a gabapentinoid.

121. The implant of claim 120, wherein the gabapentinoid is pregabalin or a pharmaceutically acceptable salt thereof.

122. The implant of claim 120, wherein the gabapentinoid is gabapentin or a pharmaceutically acceptable salt thereof.

123. The implant of claim 119, wherein the implant comprises a plurality of nanofibers comprising the IOP-lowering drug and the one or more bioresorbable polymers.

124. The implant of claim 119, wherein the implant comprises a plurality of nanoparticles comprising the IOP-lowering drug and the one or more bioresorbable polymers.

125. The implant of claim 119, wherein the implant provides zero-order release of the IOP-lowering drug.

126. The implant of claim 119, wherein the implant releases the IOP-lowering drug by diffusion through the polymer matrix.

127. The implant of claim 119, wherein the implant provides an initial burst release of the IOP-lowering drug followed by a slower sustained release of the IOP-lowering drug following implantation in the intravitreal space of the subject.

128. The implant of claim 119, wherein the one or more bioresorbable polymers comprise poly(alpha-hydroxy acids), poly(lactide-co-glycolide) (PLGA), poly(DL-lactide-co-caprolactone) (DL-PLCL), polycaprolactone (PCL), poly(L-lactic acid) (PLA), poly(trimethylene carbonate) (PTMC), polydioxanone (PDO), poly(4-hydroxy butyrate) (PHB), polyhydroxyalkanoates (PHA), poly(phosphazene), polyphosphate ester, poly(amino acid), polydepsipeptides, poly(butylene succinate) (PBS), polyethylene oxide, polypropylene fumarate, polyiminocarbonates, poly(lactide-co-caprolactone) (PLCL), poly(glycolide-co-caprolactone) (PGCL) copolymer, poly(D,L-lactic acid), polyglycolic acid, poly(L-lactide-co-D,L-lactide), poly(L-lactide-co-glycolide), poly(D,L-lactide-co-glycolide), poly(gycolide-trimethylene carbonate), poly(glycolide-co-caprolactone) (PGCL), poly(ethyl glutamate-co-glutamic acid), poly(tert-butyloxy-carbonylmethyl glutamate), poly(glycerol sebacate), tyrosine-derived polycarbonate, poly 1,3-bis-(p-carboxyphenoxy) hexane-co-sebacic acid, polyphosphazene, ethyl glycinate polyphosphazene, polycaprolactone co-butylacrylate, a copolymer of polyhydroxybutyrate, a copolymer of maleic anhydride, a copolymer of poly(trimethylene carbonate), polyethylene glycol (PEG), hydroxypropylmethylcellulose and cellulose derivatives, polysaccharides (e.g., hyaluronic acid, chitosan and starch), proteins (e.g., gelatin and collagen), polyaspirins, polyphosphagenes, collagen, alginates, albumin, fibrin, vitamin E analogs, D-lactide, D,L-lactide, L-lactide, D,L-lactide-caprolactone (DL-CL), D,L-lactide-glycolide-caprolactone (DL-G-CL), dextrans, vinylpyrrolidone, polyvinyl alcohol (PVA), PVA-g-PLGA, PEGT-PBT copolymer (polyactive), methacrylates, poly(N-isopropylacrylamide), PEO—PPO-PEO (pluronics), PEO—PPO-PAA copolymers, PLGA-PEO-PLGA, PEG-PLG, PLA-PLGA, poloxamer 407, PEG-PLGA-PEG triblock copolymers, SAIB (sucrose acetate isobutyrate) hydroxypropyl cellulose, hydroxypropyl methylcellulose, hydroxyethyl methylcellulose, carboxymethylcellulose or salts thereof, Carbopol®, poly(hydroxyethylmethacrylate), poly(methoxyethylmethacrylate), poly(methoxyethoxy-ethylmethacrylate), polymethylmethacrylate (PMMA), methylmethacrylate (MMA), gelatin, polyvinyl alcohols, propylene glycol, or a combination thereof.

129. The implant of claim 128, wherein the one or more bioresorbable polymers comprise poly(lactide-co-glycolide) (PLGA).

130. The implant of claim 119, wherein the implant provides sustained release of the IOP-lowering drug for at least 6 months in the intravitreal space of the subject.

131. The implant of claim 119, wherein the implant has one of the following shapes: spherical, oblate, ellipsoidal, rod, disc, tube, hemispherical, oval, cylindrical, or irregular.

132. The implant of claim 119, wherein the implant is sized and shaped suitably for injection through a 19G to 30G needle.

133. The implant of claim 119, wherein the implant releases the IOP-lowering drug at a rate of 0.5 μg/day to 5 μg/day in the intravitreal space of the subject.

134. The implant of claim 119, wherein the weight ratio of the IOP-lowering drug to the one or more bioresorbable polymer is from 1:1 to 1:20.

135. A biodegradable implant configured for intravitreal administration of a gabapentinoid to a subject, the implant comprising:
(i) 0.1-50 wt % of the gabapentinoid; and
(ii) 50-99.9 wt % of poly(lactic-co-glycolide) (PLGA);
wherein the one or more bioresorbable polymers form a polymer matrix containing the gabapentinoid; and
wherein the matrix provides for sustained release of the gabapentinoid in an intravitreal space of the subject.

136. The implant of claim 135, wherein the gabapentinoid is pregabalin or a pharmaceutically acceptable salt thereof.

137. The implant of claim 135, wherein the gabapentinoid is gabapentin or a pharmaceutically acceptable salt thereof.

138. The implant of claim 135, wherein the implant comprises a plurality of nanofibers comprising the gabapentinoid and the PLGA.

139. The implant of claim 135, wherein the implant comprises a plurality of nanoparticles comprising the gabapentinoid and the PLGA.

140. The implant of claim 135, wherein the implant provides zero-order release of the gabapentinoid.

141. The implant of claim 135, wherein the implant releases the gabapentinoid by diffusion through the polymer matrix.

142. The implant of claim 135, wherein the implant provides an initial burst release of the gabapentinoid followed by a slower sustained release of the gabapentinoid following implantation in the intravitreal space of the subject.

143. The implant of claim 135, wherein the implant provides sustained release of the gabapentinoid for at least 6 months in the intravitreal space of the subject.

144. The implant of claim 135, wherein the implant releases the gabapentinoid at a rate of about 0.5 to 5 μg/day in the intravitreal space of the subject.

145. The implant of claim 135, wherein the weight ratio of the gabapentinoid to the one or more bioresorbable polymer is from 1:1 to 1:20.

146. A method of treating an ocular condition in a subject in need thereof, comprising intravitreally administering to the subject the biodegradable implant of claim 119.

147. The method of claim 146, wherein the ocular condition is elevated intraocular pressure (IOP), glaucoma, intraocular inflammation, keratitis, dry eye, macular edema, diabetic macular edema (DME), infection, macular degeneration, age-related macular degeneration (AMD), blurred vision, herpetic conjunctivitis, blepharitis, retinal or choroidal neovascularization, uveitis, diabetic retinopathy, ischemic retinopathy, optic neuropathy, ocular cancer, chronic ocular allergic conditions, or cataract.

148. The method of claim 147, wherein the ocular condition is glaucoma.
